(19) United States
(12) Patent Application Publication
(10) Pub. No.: US 2009/0110694 A1
(43) Pub. Date: Apr. 30, 2009

(54) Anti-Inflammatory Proteins and Improved Vaccines

(76) Inventors: Geoffrey Smith, London (GB); An-Jung Chen, London (GB)

Correspondence Address: LEON R. YANKWICH, 201 BROADWAY, CAMBRIDGE, MA, 02139, US

(21) Appl. No.: 12/225,456

(22) PCT Filed: Mar. 22, 2007
(86) PCT No.: PCT/GB2007/001032

§ 371 Date: Dec. 11, 2008

(30) Foreign Application Priority Data
Mar. 22, 2006 (GB) 0605761.6

(51) Int. Cl. A61K 39/12; C12N 7/00; C12N 15/11; C07K 2/00; A61K 38/02; A61K 31/7088; C12N 5/06

(52) U.S. Cl. 424/199.1; 435/235.1; 536/23.72; 530/300; 514/2; 514/44; 435/375

(57) ABSTRACT

A recombinant poxvirus lacking a functional gene corresponding to BI4R in the WR strain of VACV for use as a medicament especially as a vaccine against a disease caused by a poxvirus or another pathogenic agent or for use as a medicament against a disease associated with aberrant cells. An isolated nucleotide or polypeptide encoding a poxvirus sequence corresponding to the sequence of B14 in the WR strain of VACV especially for use as a medicament against undesirable inflammation, immune activation or NF-κB activation. Related compositions and methods.

The present invention relates to anti-inflammatory proteins derived from poxviruses and to methods associated therewith. The invention further relates to improved vaccines comprising poxviruses or poxvirus vectors.

### BACKGROUND

### Poxviruses as Vaccines

In the 20^{th} century, Vaccinia virus (VACV) was used to vaccinate large numbers of humans against Variola virus (VARV) the causative agent of smallpox. The vaccination campaign was highly successful and smallpox was declared eradicated by the WHO in 1980. The success of VACV as a live vaccine was due, in part, to its low production cost, heat stability, effectiveness against all strains of VARV and the ability to administer the vaccine by simple dermal abrasion. Those advantages make VACV and other poxviruses attractive for use as the basis for recombinant viral vaccines and, consequently, VACV is currently the most explored recombinant viral vaccine (see, for example, Smith et al., 1983, Panicali et al., 1983, Moss et al., 1996, and Dorrell et al., 2001).

The genome of VACV strain Copenhagen has been sequenced (Goebel et al., 1990) and was found to comprise in the region of 200 genes. Of those genes, at least one third are dispensible for virus replication in vitro (Perkus et al., 1991). VACV possesses several genes that aid evasion or suppression of the host immune system (Seet et al., 2003).

A number of proteins are secreted from poxvirus-infected cells that can bind and inhibit specific components of the host immune system including interferons (IFNs), complement, cytokines and chemokines (Alcami and Koszinowski, 2000).

The present invention relates to a poxvirus protein that is newly characterised herein as a virulence factor and dispensible for virus replication in cell culture, and which is useful in both improved vaccines and in therapeutic modulation of the NF-κB pathway.

### NF-κB

NF-κB is a transcription factor that plays an important role in the regulation of biological systems. The biological system in which NF-κB plays the most important role is the immune system. However, NF-κB also regulates the expression of genes outside the immune system and can influence multiple aspects of normal and diseased physiology. A role for NF-κB has been recognised in embryonic development and in the development in physiology of the number of tissues including the mammary gland, bone, skin and the central nervous system. The NF-κB signalling pathway has been implicated in a number of diseases and disorders and particularly in inflammatory and autoimmune diseases. Components of the NF-κB signalling pathway, including NF-κB itself, are therefore regarded as potentially good drug targets for therapeutic intervention in such diseases.

The present invention relates to a protein found in VACV and other related poxviruses that can be used in order to influence the NF-κB pathway.

### The NF-κB Pathway

There are five known members of the mammalian NF-κB family, p65 (RelA) RelB, c-Rel, p50/p105 (NF-κB1) and p52/p100 (NF-κB2). NF-κB family members exist in the cytoplasm of unstimulated cells as homo- or hetero-dimers bound to IκB family members. Binding of IκB prevents the NF-κB-IκB complex from translocating to the nucleus and thereby maintains NF-κB in an inactive state. When NF-κB is activated by dissociation from IκB it can translocate to the nucleus where it can bind to promoter or enhancer regions of the genomic DNA thereby influencing gene expression. There are two pathways by which NF-κB may be activated, known as the classical pathway and the alternative pathway. In the classical pathway of NF-κB activation, activation is triggered by increased activity of the Beta subunit of the IκB kinase complex (IKK complex) that then phosphorylates IκB on two N-terminal serine residues. In the alternative pathway, IKKα is activated and phosphorylates p100. In both pathways phosphorylated IκBs are recognised by the ubiquitin ligase machinery leading to their polyubiquitination and subsequent degradation or processing by the proteasome. The degradation or processing of IκB frees the NF-κB hetero-dimers to translocate to the nucleus where they bind to specific sequences in the promoter or enhancer regions of target genes. Activated NF-κB is down-regulated by various mechanisms including a feedback pathway whereby newly synthesised IκB protein binds to nuclear NF-κB and re-exports it out to the cytosol. There are seven known IκB family members, IκBα, IκBβ, BCL-3, IκBv, IκBγ and the precursor proteins p100 and p105, which are characterised by the presence of 5 to 7 ankyrin repeats that assemble into elongated cylinders that bind to the dimerisation domain of NF-κB dimers.

### Signalling Pathways to NF-κB

A number of pathways lead to the activation of NF-κB, almost always by the activation of IKK, degradation of IκB, and enhancement of the transcriptional activity of NF-κB. Activation of the tumour necrosis factor receptor (TNF-R), the toll-like-receptors (TLRs), the T-cell receptor (TCR) and B-cell receptor (BCR) all lead to NF-κB activation. Other proteins that signal to NF-κB include lymphotoxin-β receptor, BAFF-R and CD40. Additionally, intracellular stresses both physiological and pathological induce the activation of NF-κB. A properly orchestrated response to genotoxic stress is a fundamental defence against cellular transformation and against cancer. The NF-κB-mediated response to genotoxic stress in some circumstances can be a provision of an anti-apoptotic signal thereby providing a window of opportunity for the cell to carry out DNA repair. Under other circumstances genotoxic stress causes NF-κB signalling that results in a pro-apoptotic response. The NF-κB pathway has been implicated in the pathogenesis of several diseases including lymphoid cancers particularly Epstein-Barr virus (EBV)-positive Hodgkins lymphomas and HTLV-III positive lymphomas. NF-κB is also thought to play an important role in antigen specific proliferation of immune effector cells, thereby suggesting that disruption of the NF-κB pathway may be beneficial in the treatment of antigen specific autoimmunity. More generally the NF-κB pathway is likely to be involved in any disease or pathological process that has been linked to an aberrant inflammatory response.

### NF-κB Inhibitors

A number of drug compositions have been proposed that are thought to act through inhibition of NF-κB, including the composition disclosed in EP1541139.

### Virulence Factors in Vaccinia Virus

VACV is a member of the Orthopoxvirus genus of the Poxyiridea a group of large double stranded DNA viruses replicating in the cytoplasm. The VACV strain Copenhagen genome (191 kb) was predicted to encode around 200 genes (Goebel et al., 1990). The central region of the genome (˜100 kb) is highly conserved and contains 90 genes that are present in all sequenced chordopoxviruses (Upton et al., 2003; Gubser et al., 2004). Most of these genes encode proteins with essential functions for viral replication. In contrast, genes located in the terminal regions are more variable and encode proteins that are non-essential for virus replication but which affect virus virulence, host range and modulation of the host response to infection (Smith & McFadden, 2002). Immunomodulators from VACV and other poxviruses may act inside or outside the infected cell and may block the action of cytokines, chemokines and IFNs or intracellular signalling pathways leading to apoptosis or gene activation (Seet et al., 2003). Gene B14R of VACV strain Western Reserve (WR) is located in the right terminal region that is rich in immunomodulators. For instance, the upstream gene encodes an intracellular protein that inhibits caspase-1 activity and thereby induction of apoptosis (Dobbelstein & Shenk, 1996; Kettle et al., 1997), and the downstream gene encodes a soluble interleukin (IL)-11 binding protein (Alcami & Smith, 1992; Spriggs et al., 1992) that inhibits development of fever during systemic infection (Alcami & Smith, 1996). VACV strain WR gene B14R is predicted to encode a 149 amino acid protein (Howard et al., 1991; Smith et al., 1991). The equivalent gene in VACV strain Copenhagen is called B15R due to disruption of the preceding open reading frame (ORF) into two fragments called B13R and B14R (Gubser & Smith, 2002; Gubser et al., 2004). Hereinafter, the gene will be referred to as B14R, which will be taken to mean the gene corresponding to the B14R gene in VACV strain WR and related strains and to the B15R gene in the Copenhagen strain of VACV. The encoded proteins are known as B14 in the WR strain of VACV and B15 in the Copenhagen strain of VACV. Likewise, the encoded protein will hereinafter be referred to as B14, which will be taken to mean the B14 protein in VACV strain WR and related strains and the B15 protein in the Copenhagen strain of VACV. Unusually for a gene located in a terminal region of the VACV genome, VACV WR B14R is highly conserved among different orthopoxviruses, suggesting an important function (Gubser & Smith, 2002; Gubser et al., 2004). Although the B14R gene is conserved, hitherto there has been no characterisation of the encoded protein or functional analysis of viruses lacking this gene.

### SUMMARY OF THE INVENTION

The invention provides a recombinant poxvirus, wherein the poxvirus genome does not comprise a functional gene corresponding to B14R in the WR strain of VACV, for use as a medicament.

The invention provides a vaccine composition comprising a poxvirus according to the invention and a pharmaceutically suitable carrier.

The invention provides a vaccine kit comprising a composition according to the second aspect.

The invention provides a method of vaccinating a subject comprising administering to the subject an immunogenic agent, wherein the immunogenic agent is poxvirus as defined according to the invention or vaccine composition according to the invention.

The invention provides use of a recombinant poxvirus having a genome that does not comprise a functional gene corresponding to B14R in the WR strain of a VACV for the manufacture of a vaccine for the immunoprophylaxis of an infection caused by a poxvirus.

The invention provides a recombinant poxvirus having a genome comprising a non-poxvirus gene or a fragment of a non-poxvirus gene which gene or fragment encodes an antigen, wherein the poxvirus genome does not comprise a functional gene corresponding to B14R in WR strain of a VACV.

The invention provides use of a recombinant poxvirus according to the invention for the manufacture of a vaccine for the prophylaxis of an infection caused by a pathogenic agent wherein the poxvirus has a genome comprising a non-poxvirus gene or a fragment of a non-poxvirus gene which gene or fragment encodes an antigen of the pathogenic agent.

The invention provides use of a recombinant poxvirus according to the invention for the manufacture of a vaccine for the prophylaxis or treatment of a disease associated with aberrant cells, wherein said poxvirus has a genome comprising a non-poxvirus gene or a fragment of a non-poxvirus gene which gene or fragment encodes an antigen of the aberrant cells comprising the gene product of the said non-poxvirus gene.

The invention provides an isolated nucleic acid molecule comprising a nucleotide sequence corresponding to a poxvirus gene corresponding to B14R in the WR strain of a VACV or a functional fragment or derivative thereof.

The invention provides an isolated polypeptide molecule comprising an amino acid sequence corresponding to a poxvirus protein corresponding to B14 in the WR strain of a VACV or a functional fragment or derivative thereof.

The invention provides an isolated nucleic acid molecule, fragment or derivative or an isolated polypeptide molecule, fragment or derivative according to the invention for use as a medicament.

The invention provides use of an isolated nucleic acid molecule fragment or derivative according to the invention or an isolated polypeptide molecule, fragment or derivative, according to the invention in the manufacture of a medicament for treatment of a disease or disorder characterized by undesirable inflammation.

The invention provides use of an isolated nucleic acid molecule, fragment or derivative, according to the invention or an isolated polypeptide molecule, fragment or derivative, according to the invention in the manufacture of a medicament for the treatment of a disease or disorder characterized by undesirable NF-κB activation.

The invention provides use of an isolated nucleic acid molecule, fragment or derivative, according to the invention or an isolated polypeptide molecule, fragment or derivative, according to the invention in the manufacture of a medicament for treatment of a disease or disorder characterized by undesirable inflammation.

The invention provides a pharmaceutical composition comprising an isolated nucleic acid molecule, fragment or derivative, according to the invention or an isolated polypeptide molecule, fragment or derivative, according to the invention and a pharmaceutical carrier.

The invention provides a method of inhibiting NF-κB activation in cells comprising providing cells with an effective amount of an isolated polypeptide molecule, fragment or derivative, according to the invention.

The invention provides a method of treating a disease or disorder characterized by undesirable NF-κB activation, comprising administering a therapeutically effective amount of an isolated nucleic acid molecule, fragment or derivative according to the invention or an isolated polypeptide molecule, fragment or derivative, according to the invention or a pharmaceutical composition according to the invention to a subject in need of said treatment.

The invention provides a method of treating a disease or disorder characterized by undesirable inflammation, comprising administering a therapeutically effective amount of an isolated nucleic acid molecule, fragment or derivative, according to the invention or an isolated polypeptide molecule, fragment or derivative, according to the invention or a pharmaceutical composition according to the invention to a subject in need of said treatment.

The invention provides a method of treating a disease or disorder characterized by undesirable immune activation, comprising administering a therapeutically effective amount of an isolated nucleic acid molecule, fragment or derivative, according to the invention or an isolated polypeptide molecule, fragment or derivative, according to the invention or a pharmaceutical composition according to the invention to a subject in need of said treatment.

### BRIEF DESCRIPTION OF THE FIGURES

FIG. 1. A rooted phylogenetic tree of VACV WR B14 and related poxvirus proteins. The sequences of the individual proteins were aligned using programme ClustalW and a rooted tree was derived from this alignment using PHYLIP—Phylogeny Inference Package (Version 3.2) via the Biology WorkBench 3.2 website (See workbench.sdsc.edu). The bootstrap values from 1000 replica samplings are indicated. See www.poxvirus.org for details of B14 protein related proteins.

FIG. 2. Characterization of VACV WR B14 by immunoblotting. (a) Wild type and C-terminally VACV HA-tagged WR B14 were expressed transiently in HeLa cells. At 24 h p.i. extracts of cells were prepared using RIPA buffer (20 mM Tris.HCl, pH 8.0, 150 mM NaCl, 1% Nonidet P-40, 1% SDS) and analysed by immunoblotting with either rabbit anti-B14 Ab (left) or an anti-HA mAb (right). (b) HeLa cells were infected by the indicated VACVs at 10 p.f.u./cell for 16 h, or mock infected or transfected with pCl-B14 for 24 h. The cells were lysed in RIPA buffer and extracts were analysed by immunoblotting with anti-D8 mAb (top panel) or anti-B14 Ab (lower panel). (c) HeLa cells were infected with VACV WR at 10 p.f.u./cell in the presence (+) or absence (−) of 40 μg ml⁻¹ cytosine arabinoside (AraC). At the indicated times p.i. the cells were washed once with PBS and lysed by addition of 200 μl of protein loading buffer. Twenty μl of each cell lysate was resolved by SDS-PAGE and analysed by immunoblotting with ant-D8 mAb (top panel) or anti-B14 Ab (lower panel). In all panels the sizes of molecular mass markers are indicated in kDa.

FIG. 3. Subcellular localisation of WR B14. (a) WR B14 with a C-terminal HA tag was expressed transiently by transfection in HeLa cells for 24 h. Cells were fixed and permeabilised as described (in the examples) and stained with anti-HA mAb (left panels), anti-B14 Ab (top right panel) or rabbit pre-immune serum Abs. (b) HeLa cells were infected with vB14 (top panels) or vΔB14 (lower panels) at 10 p.f.u./cell for 8 h. Cells were fixed and permeabilised as in (a) and stained with either anti-D8 mAb (left panels, green in original) or anti-B14 Ab (right panels, red in original).

FIG. 4. Plaque formation by WR vΔB14. Monolayers of CV-1, RK₁₃ or BS-C-1 cells were infected by WR vB14, WR vΔB14 or WR vB14-rev and overlaid with DMEM/2.5% FBS/1% carboxymethyl cellulose. After 48 (CV-1 & BSC-1 cells) or 72 h (RK₁₃ cells), the monolayers were stained with 0.1% crystal violet in 15% ethanol and photographed.

FIG. 5. Growth kinetics of WR vΔB14. BS-C-1 cells were infected at either 10 (a) or 0.01 (b) p.f.u./cell and aliquots of infected cells were collected at the indicated times p.i. Cells were frozen and thawed three times, sonicated and the virus infectivity was titrated in duplicate on BS-C-1 cell monolayers. The data are presented as the mean log 10 p.f.u.±SD. Data for WR vB14 and WR vB14-rev also shown.

FIG. 6. Virulence assay in murine i.d. model. (a) Female C57BL/6 mice (n=6) were infected i.d. with 10⁴ p.f.u. of the indicated viruses and the size of the lesion was measured daily with a micrometer. Horizontal bar indicates the days on which the lesion size caused by WR vΔB14 was statistically different (P=<0.05) from both WR vB14 and WR vB14-rev. Data are means±SD of lesion size. (b) Infectious virus in infected lesions. Mice were infected in both ears as in (a) and at the indicated time p.i. the animals were sacrificed, ears were removed and infectious virus was determined by plaque on assay on duplicate monolayers of BS-C-1 cells. Asterisks indicate days on which the titre of virus present in ears infected with WR vΔB14 was significantly different from both WR vB14 and WR vB14-rev infected tissue. Data are means±SD of viral titres.

FIG. 7. Characterisation of infiltrating leukocytes in the C57BL/6 mice infected intradermally with 10⁴ p.f.u. of the indicated viruses. Cells were extracted (Methods) from both ears of an infected mouse and were pooled. (a) Total numbers of viable cells were determined by trypan blue exclusion. Data are means±SD of cell counts. In these cells, neutrophils (b), macrophages (c) and T cells (d) were identified by corresponding surface markers as described in Methods. The absolute number of neutrophils (e), macrophages (f) and T cells (g) at days 8 and 11 p.i. were determined as described in Methods. The means±SD of data from two infected mice were analysed as a group at indicated times by staining and flow cytometry. Asterisks indicate the days on which the data for WR vΔB14 are significantly different from both WR vB14 and WR vB14-rev groups (P<0•05, Student's t-test).

FIG. 8. Analysis of recombinant WR VACV genomes by PCR. Virus DNA was extracted from cells infected with the indicated viruses or mock infected and used as template for PCR using L-FA and R-FB primers (see method section of examples) flanking the B14R gene locus. PCR products were analysed by agarose gel electrophoresis. Arrows indicate the size of DNA fragments expected for the wild type or deleted B14R gene alleles.

FIG. 9. Characterisation of the WR VACV genomes. DNA was extracted from purified intracellular mature virus (IMV) for the indicated viruses and 10 μg of each DNA was digested by SphI and the resulting DNA fragments were resolved on a 0.8% agarose gel containing ethidium bromide. Arrows indicated the position of DNA fragments A (2.9 kb) and B (2.5 kb) containing the wild type or deleted B14R alleles, respectively. A diagrammatic representation of the genome around the B14R gene is shown with the SphI cleavage sites marked. The parental virus (Parental vB14) was the initial viral stock to generate recombinant viruses.

FIG. 10. Growth kinetics of vΔB14. CV-1 cells were infected at either 10 (a) or 0.01 (b) p.f.u./cell and aliquots of infected cells were collected at the indicated times p.i. Cells were frozen and thawed three times, sonicated and the virus infectivity was titrated in duplicate on BS-C-1 cell monolayers. The data are presented as the mean log 10 p.f.u.±SD.

FIG. 11. The WR VACV B14 protein did not affect virus virulence in a murine intranasal model under the conditions tested. (a) Mice were infected intranasally with 10⁴ (n=3) (a) or 3×10³ (n=5) (b) p.f.u: of vB14, vΔB14 or vB14-rev. The body weight of each mouse was monitored daily. The mean weight of each group of animals on each day is expressed as a percentage of the mean weight of the same group of animals on day zero. The data are presented as the mean weight±SD.

FIG. 12. HeLa cells were co-transfected with plasmids encoding NF-κB-luciferase, β-galactosidease and WR B14 (a) or WR B14 or WR B14-HA (b) as indicated. At 24 h following transfection, cells were stimulated with 100 ng/ml of IL-1, or TNF-α for 8 h. Luciferase activity was then measured in triplicate wells. Data were normalized by β-galactosidase intensity in the same well and are expressed as the mean fold induction compared to the average of normalised luminescence unit value in the pCl-transfected cells. Asterixes indicate significant difference compared to pCl-transfected cells under stimulation. Data are means±SD of the induction. (P<0.05, Student's t-test).

FIG. 13. HeLa cells were transfected with an NF-κB-luciferase reporter plasmid for 24 h and subsequently were infected by indicated VACV at 1 p.f.u./cell. At 2 h p.i., the infected cells were stimulated with 100 ng/ml of IL-1β for 6 h and cell extracts prepared (see examples). The luciferase activity was normalised by the total protein content from the corresponding extract as a transfection efficiency control. Data are expressed as the mean fold induction as a ratio of the mean of the normalised luciferase activity in the mock infection (Student's t-test: * P<0.05: ** P<0.005).

FIG. 14. Extracts from HeLa cells mock-infected or infected with the indicated VACVs were either mock-stimulated (−) or stimulated (+) with 20 μg/ml or IL-1β for 20 min. The cytosolic proteins (15 μg per lane) were resolved by SDS-PAGE and analysed by immunoblotting with the Abs indicated to the left of the figure.

FIG. 15. Extracts from HeLa cells infected with the indicated VACVs were immunoprecipitated with anti-HA mAb. The immunoprecipitated proteins were resolved by SDS-PAGE and analysed by immunoblotting using Abs indicated on the left. The position of the immunoglobulin heavy chain is indicated to the right of the figure by arrows.

FIG. 16. Extracts from HeLa cells infected with the indicated VACVs were immunoprecipitated with anti-HA, anti-IKKα or IKKβ Abs. The immunoprecipitated proteins were resolved by SDS-PAGE and were analysed by immunoblotting with the Abs indicated on the left of the figure. The position of the immunoglobulin (Ig) heavy chain is indicated on the right.

FIG. 17. A cytoplasmic extract from HeLa cells infected with VACV WR vB14 was subjected to gel filtration on a Superose 6 column. Fractions were resolved by SDS-PAGE and analysed by immunoblotting with the indicated Abs. The position of protein size markers is indicated in kDa at the top of the figure.

### DETAILED DESCRIPTION OF THE INVENTION

The data presented herein provides strong evidence that B14 of the WR strain of VACV, and corresponding proteins in other poxviruses, are virulence factors that act by inhibiting the activation of the NF-κB pathway. This characterisation suggests uses in two technical fields. The first technical field relates to the production of improved vaccines using poxviruses that either lack B14 or its equivalent or have a non-functional derivative, which will be more effective because the absence of an inhibitor of NF-κB activation will permit more effective activation of immune effector cells. The second technical field relates the production of therapeutic agents derived from B14, or its equivalents, which act to inhibit undesirable NF-κB activation.

The invention provides a recombinant poxvirus, wherein the poxvirus genome does not comprise a functional gene corresponding to B14R in the WR strain of VACV, for use as a medicament.

Said recombinant poxvirus may be used as a vaccine against a disease caused by a poxvirus. Examples of diseases caused by poxviruses include human and animal poxvirus diseases such as smallpox (which is at risk of being reintroduced as a biological weapon through use in war or by terrorism), monkeypox, cowpox, camelpox, fowipox, sheeppox, Orf and swinepox. The recombinant poxvirus may also be an attenuated poxvirus, for example for use as an attenuated vaccine to protect against complications induced by vaccination with wild type VACV.

Vaccination with a given poxvirus is cross protective against poxviruses of the same genus. Accordingly, vaccination with VACV provides protection against variola virus, monkeypox virus and cowpox virus. Vaccination with other poxviruses provides protection against poxviruses of the respective homologous genus.

Preferably, the recombinant poxvirus is an orthopoxvirus or a derivative thereof. The recombinant poxvirus may be a VACV, a cowpox virus, a monkeypox virus, a camelpoxvirus or an ectromelia virus or a derivative of any of those viruses.

Preferably, the recombinant poxvirus is a VACV.

Preferably, the recombinant poxvirus is a VACV selected from a group consisting of Lister, Copenhagen, WR, Wyeth, New York City Board of Health, NYVAC, Praha virus, DRYVAX Wyeth-derived virus, LIVP, IHD-J, IHD-W, Tian Tan, Tashkent, King Institute, Patwadanger, EM-63, Evans, Bern, LC16 m8 or MVA. More preferably the recombinant poxvirus is a VACV strain selected from the group consisting of MVA, Lister, Copenhagen and Wyeth.

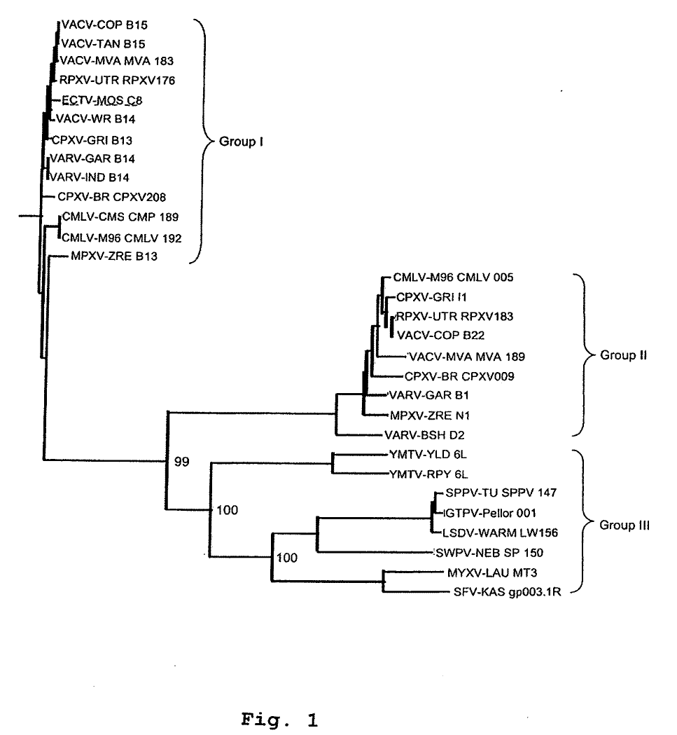

The recombinant poxvirus is selected from a strain that normally has a gene corresponding to B14R in the WR strain of VACV. By “corresponding” it is meant that the gene is functionally equivalent to B14R in that it encodes a polypeptide having a similar activity to B14 (for example the ability to bind to the IKK complex or the ability to inhibit activation of the NF-κB pathway as measured in one of the methods outlined in the Examples herein). An alternative definition of a “corresponding” gene is a gene that shares at least, 40%, at least 50%, at least 55%, at least 60%, at least 65%, at least 70%, at least 75%, at least 80%, at least 82%, at least 85%, at least 87%, at least 90%, at least 92%, at least 93%, at least 94%, at least 95%, at least 96%, at least 97%, at least 98%, or at least 99% identity at either the amino acid level or alternatively at the nucleotide level, as judged by a sequence alignment tool such as BLAST (NCBI, NLM, NIH). An alternative definition of “corresponding” gene is a gene that is phylogenetically closely related to B14R (for example which shares a close common ancestral gene in viral evolution). Examples of genes that are is phylogenetically closely related to B14R include the genes that give rise to the poxvirus proteins illustrated in FIG. 1 as their gene products.

Preferably the recombinant poxvirus of the invention is for use as a vaccine against a disease caused by an orthopoxvirus infection in a human. Said disease may be a disease may be smallpox, monkeypox or cowpox. Preferably said disease is smallpox. It may also be a disease caused by a biological weapon comprising a virus derived from an orthopoxvirus, for example from VARV, said derivative virus being artificially derived.

The invention finds application in the veterinary field. The recombinant poxvirus may be for use as a vaccine against a disease caused by an orthopoxvirus infection in an animal, in particular in a mammal, for example, a non-rodent mammal. The animal may be, for example, a companion animal, an animal used in animal husbandry, or an animal used in sport or for transport, for example, a cat or dog, a member of the cattle family, a sheep or goat, a pig, a horse, or a member of the camel family. The disease caused by an orthopoxvirus in an animal is especially one selected from the group consisting of monkeypox, cowpox, and camelpox. For example, the medicament may be for use in the immunisation of cats against cat pox, mice against ectromelia, members of the camel family against camelpox, a wide range of mammals, for example rodents, cats, cows and other cattle, large felines or elephants against cowpox, or rodents or monkeys against monkeypox.

A recombinant poxvirus of the invention may be selected from the group consisting of parapoxviruses, avipoxviruses, suipoxviruses, molluscipoxviruses and yatapoxviruses.

The recombinant poxvirus of the invention may be used against a disease caused by a poxvirus infection in a human or alternatively against a disease caused by a virus infection in a human, said virus infection not being a poxvirus infection.

A recombinant poxvirus of the invention may have no coding sequence corresponding to the sequence of B14R in the WR strain of VACV. That is to say the recombinant poxvirus will have had the complete coding sequence deleted. Alternatively, the coding sequence may have been merely disrupted, mutated or truncated such that its gene product has reduced inhibitory activity (or example reduced activity as judged in one of the assays disclosed in the Examples herein). Alternatively, the activity of the gene may be reduced by the introduction of one or more mutations or deletions in the gene's promoter or other upstream expression controlling regions so as to compromise expression of the gene leading to reduced levels of gene expression. Preferable levels of gene product activity or levels of gene expression are reduced by over 50%, by over 70%, or more preferably by over 80% or most preferably by over 90% compared to activity or expression levels in a poxvirus which has not had the activity or expression of the gene reduced. According to certain embodiments of the invention, the recombinant poxvirus genome comprises a non-poxvirus gene or a fragment of a non-poxvirus gene, which gene or fragment encodes an antigen.

The recombinant poxviruses of the invention may be made by any suitable technique of genetic engineering. For example, homologous recombination and PCR techniques. Examples of suitable techniques are given in the Example section herein.

The invention also provides a vaccine composition comprising a recombinant poxvirus of the invention and a pharmaceutically suitable carrier.

The vaccine composition may comprise one or more additives selected from the group comprising an antibiotic, a preservative, a stabiliser and an adjuvant. The immunising effect of an immunogen in a vaccine may be enhanced by the addition of an adjuvant. An adjuvant co-stimulates the immune system in an unspecific manner causing a stronger specific immune reaction against the immunogenic determinant in the vaccine. Preferably said vaccine composition is sterile except for the vaccine component.

The invention provides a vaccine kit comprising a composition according to the second aspect.

The invention further provides a method of vaccinating a subject comprising administering to the subject an effective amount of an immunogenic agent, wherein the immunogenic agent is a poxvirus according to the invention or a vaccine composition according to the invention. The vaccination generally induces an immune response to the poxvirus used as immunogenic agent and hence provides protection against infection by the poxvirus or an immunogenically cross-reacting poxvirus.

The term “subject” is used herein to denote a human or a non-human animal. A non-human animal is, in particular, a mammal and may be a non-rodent mammal. The animal may be, for example, a companion animal, an animal used in animal husbandry or an animal used in sport or for transport, for example, a cat or dog, a member of the cattle family, a sheep or goat, a pig, a horse, or a member of the camel family.

The term “effective amount” denotes an amount effective to achieve the desired result.

The invention provides use of a recombinant poxvirus having a genome that does not comprise a functional gene corresponding to B14R in the WR strain of VACV for the manufacture of a vaccine for the immunoprophylaxis of an infection caused by a poxvirus.

The invention provides a recombinant poxvirus having a genome comprising a non-poxvirus gene or a fragment of a non-poxvirus gene which gene or fragment encodes an antigen, wherein the poxvirus genome does not comprise a functional gene corresponding to B14R in WR strain of VACV.

The invention provides use of a recombinant poxvirus according to the invention for the manufacture of a vaccine for the prophylaxis of an infection caused by a pathogenic agent wherein the poxvirus has a genome comprising a non-poxvirus gene or a fragment of a non-poxvirus gene which gene or fragment encodes an antigen of the pathogenic agent.

By “non-poxvirus gene” is meant herein a gene not belonging to a poxvirus of the genus of the poxvirus in question. For a given recombinant poxvirus, the non-poxvirus gene may be a gene belonging to a poxvirus of a different genus. Preferably, the non-poxvirus gene is a gene not belonging to any poxvirus.

The recombinant poxvirus of the invention is preferably for use as a vaccine for the prophylaxis of an infection caused in a subject by a pathogenic agent.

Preferably, the gene or gene fragment that encodes an antigen may be any non-poxvirus gene or gene fragment against the gene product of which a cellular immune response in a subject is desirable. Suitable genes include those encoding immunogenic peptides or polypeptides of an infectious pathogen, for example, for use in humans, an influenza virus (for example an avian influenza virus), malaria, HIV, heptitis C virus, hepatitis B virus, herpes virus, a parasitic pathogen, for example tuberculosis or Leishmaniasis, a protozoan, for example a protozoan that causes ameobic dysentery. For use in animals, appropriate pathogen immunogenic peptides are known to those skilled in the art.

The recombinant poxvirus of the invention may be for use as a vaccine for the prophylaxis or treatment of a disease associated with aberrant cells. In such a recombinant poxvirus, the gene or gene fragment that encodes an antigen may be any non-poxvirus gene or gene fragment encoding an antigenic peptide or an antigenic polypeptide of aberrant cells, for example cancer cells, the elimination or induced quiescence of which is beneficial. According to certain aspects of that embodiment of the antigen is a tumour-associated antigen.

In some instances, it is beneficial for the whole gene that encodes an antigen to be present in the virus. In some cases, however, a fragment of the gene will suffice. In the case of a gene fragment, a gene product smaller than that of the whole gene is produced, which smaller gene product is or comprises an epitope or epitopes of the antigen in question.

A recombinant poxvirus of the invention may be prepared by methods known in the art (see for example Boyle, D. B. and Coupar, B. E. H., Gene, 1988, 65, 123-128). For example, a poxvirus lacking a functional gene corresponding to B14R of the WR strain of VACV may be produced by transfection of cells that had previously been infected with a poxvirus with a plasmid, the plasmid comprising DNA sequences homologous to sequences of the poxvirus flanking the gene corresponding to B14R together with a selectable marker. After transfection and virus multiplication, recombinant viruses are selected using the selectable marker.

As mentioned above, the poxvirus of the invention may comprise a gene that encodes an immunogen. The gene encoding the immunogen is introduced into the poxvirus in a manner known in the art. For example, a plasmid may be used that comprises the gene together with DNA sequences homologous to sequences of the poxvirus genome such that homologous recombination can take place between the plasmid and the genomic DNA as described in U.S. Pat. No. 6,998,252. Preferably, the sequence of the poxvirus genome is one that may be disrupted without compromising the viability of the poxvirus. The homologous DNA sequences are preferably of sufficient length to enable homologous recombination between the plasmid DNA and the virus genomic DNA to take place. Accordingly, the DNA sequences preferably have a length of from 20 to 1000 bp. More preferably, the sequences have a length of from 100 to 800 bp. Still more preferably, the sequences have a length of from 300 to 500 bp. Sequences are considered homologous if they have 85% or more sequence identity. Preferably, homologous sequences have 90% or more sequence identity, more preferably, homologous sequences have 95% or more sequence identity, for example, 98% or more sequence identity. Most preferably, the homologous sequences used are identical to the genomic sequences in the virus.

By use of a plasmid comprising the gene encoding the antigen flanked by DNA sequences with homology to sequences of the poxvirus flanking the gene corresponding to B14R, it is possible to carry out the disruption of the gene that encodes B14 and the introduction of the immunogen gene in a single step.

The gene encoding the immunogen of interest is preferably engineered to be associated with transcriptional regulatory sequences for expression of the gene by poxvirus in an infected host cell. Such regulatory sequences preferably include a promoter from a poxvirus and a poxvirus termination sequence. Most preferably, a promoter from VACV or a related poxvirus and a VACV termination sequence are included. Most preferably, the VACV termination sequence is a VACV early transcriptional termination sequence (TTTTTNT).

For the preparation of a vaccine, the poxvirus according to the invention is typically provided in a physiologically acceptable form. A person skilled in the art will be familiar with suitable poxvirus vaccine formulations given the large body of knowledge that was built up in the many years of use of VACV in the vaccination against smallpox. For example, an appropriate number of particles of the recombinant poxvirus, e.g. 10⁴ to 10⁹ particles, are freeze dried in an appropriate volume, e.g. approximately 100 μl, of phosphate-buffered saline (PBS) in the presence of peptone and human albumin in, for example, a vial or an ampoule, preferably a glass ampoule. The lyophilisate can contain extenders, for example mannitol, dextran, sugar, glycine, lactose or polyviylpyrrolidone, or other excipients, for example antioxidants or stabilisers, suitable for parenteral administration. The vial or ampoule may then be sealed and may be stored for several months, preferably at a temperature below −20° C.

For vaccination, the lyophilisate may, for example, be made up to 0.1 to 0.2 ml of aqueous solution, preferably with physiological saline, and administered parenterally, for example by intradermal inoculation or by dermal abrasion. The vaccine of the invention may be infected intracutaneously. The mode of administration, the dose and the number of administrations can be optimized by those skilled in the art in a conventional manner. A high degree of immunity against the antigen is obtained by administration of the vaccine several times over a lengthy period of time.

The invention encompasses derivatives of the viruses in accordance with the invention. By “derivative” of a particular virus is meant any virus that is derived from the particular virus. A derivative may be obtained by repeated passaging of the particular virus. Alternatively, a derivative may be obtained by site directed or random mutagenesis of the particular virus. A derivative generally has most of the characteristics and most of the genes of the particular virus from which it is derived. Typically, its genome has 90% sequence identity with the genome of the particular virus. For example its genome has 95%, optionally 98% sequence identity with the genome of the particular virus.

The invention provides an isolated nucleic acid molecule comprising a nucleotide sequence corresponding to a poxvirus gene corresponding to B14R in the WR strain of VACV or a functional fragment thereof.

The invention also encompasses: (a) DNA vectors that contain a B14 coding sequences and/or their complements (i.e., antisense); (b) DNA expression vectors that contain a B14 coding sequences operatively associated with a regulatory element that directs the expression of the coding sequences (for example, baculovirus as described in U.S. Pat. No. 5,869,336 herein incorporated by reference); and (c) genetically engineered host cells that contain a B14 coding sequences operatively associated with a regulatory element that directs the expression of the coding sequences in the host cell. As used herein, regulatory elements include, but are not limited to, inducible and non-inducible promoters, enhancers, operators, and other elements known to those skilled in the art that drive and regulate expression.

Such regulatory elements include, but are not limited to, the human cytomegalovirus (hCMV) immediate early gene, regulatable, viral elements (particularly retroviral LTR promoters), the early or late promoters of SV40 or adenovirus, the lac system, the trp system, the TAC system, the TRC system, the major operator and promoter regions of phage lambda, the control regions of fd coat protein, the promoter for 3-phosphoglycerate kinase (PGK), the promoters of acid phosphatase, and the promoters of the yeast α-mating factors.

The invention provides an isolated polypeptide molecule comprising an amino acid sequence corresponding to a poxvirus protein corresponding to B14R in the WR strain of VACV or a functional, fragment or derivative, thereof.

The present invention also encompasses antibodies and anti-idiotypic antibodies (including Fab fragments), antagonists and agonists of B14, as well as compounds or nucleotide constructs that inhibit expression of a B14 sequence (transcription factor inhibitors, antisense and ribozyme molecules, or open reading frame sequence or regulatory sequence replacement constructs), or promote the expression of a B14 (e.g., expression constructs in which B14 coding sequences are operatively associated with expression control elements such as promoters, promoter/enhancers, etc.).

The invention provides an isolated nucleic acid molecule, derivative or fragment, of the invention or an isolated polypeptide molecule, derivative or fragment, of the invention for use as a medicament.

The invention provides use of an isolated nucleic acid molecule, derivative or fragment, according to the invention or an isolated polypeptide molecule, derivative or fragment, according to the invention in the manufacture of a medicament for treatment of a disease or disorder characterized by undesirable inflammation.

The invention provides use of an isolated nucleic acid molecule, derivative or fragment, according to the invention or an isolated polypeptide molecule, derivative or fragment, according to the invention in the manufacture of a medicament for the treatment of a disease or disorder characterized by undesirable NF-κB activation. Such diseases include asthma, rheumatoid arthritis, inflammatory bowel disease and cancer; see review by Yamamoto and Gaynor, Current Molecular Medicine, 2001, 1, 287-296).

The invention provides use of an isolated nucleic acid molecule, derivative or fragment, according to the invention or an isolated polypeptide molecule, derivative or fragment, according to the invention in the manufacture of a medicament for treatment of a disease or disorder characterized by undesirable inflammation. Such diseases include asthma, rheumatoid arthritis, inflammatory bowel disease, cancer, chronic obstructive pulmonary disease and multiple sclerosis.

The invention provides a pharmaceutical composition comprising an isolated nucleic acid molecule, derivative or fragment, according to the invention or an isolated polypeptide molecule, derivative or fragment, according to the invention and a pharmaceutical carrier.

The invention provides a method of inhibiting NF-κB activation in cells comprising providing cells with an effective amount of an isolated polypeptide molecule, derivative or fragment, according to the invention. The isolated polypeptide molecule may be provided directly (i.e. delivered to the cells as a polypeptide molecule). Alternatively it may be provided by introduction to the cell of a nucleic acid construct that directs expression of the polypeptide molecule.

The invention provides a method of treating a disease or disorder characterized by undesirable NF-κB activation, comprising administering a therapeutically effective amount of an isolated nucleic acid molecule, derivative or fragment, according to the invention or an isolated polypeptide molecule, derivative or fragment, according to the invention or a pharmaceutical composition according to the invention to a subject in need of said treatment.

The invention provides a method of treating a disease or disorder characterized by undesirable inflammation, comprising administering a therapeutically effective amount of an isolated nucleic acid molecule, derivative or fragment, according to the invention or an isolated polypeptide molecule, derivative or fragment, according to the invention or a pharmaceutical composition according to the invention to a subject in need of said treatment.

The invention provides a method of treating a disease or disorder characterized by undesirable immune activation, comprising administering a therapeutically effective amount of an isolated nucleic acid molecule, derivative or fragment, according to the invention or an isolated polypeptide molecule, derivative or fragment, according to the invention or a pharmaceutical composition according to the invention to a subject in need of said treatment.

Nucleic acid molecules and derivative or fragments thereof according to the invention may be used in gene therapy. Gene therapy refers to therapy performed by the administration of a nucleic acid to a subject. In this embodiment of the invention, the nucleic acid produces its encoded protein that mediates a therapeutic effect by inhibiting NF-κB function.

Any of the methods for gene therapy available in the art can be used according to the present invention. Example methods are described below.

For general reviews of the methods of gene therapy, see Goldspiel et al., 1993,

Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Moran and Anderson, 1993, Ann. Rev. Biochem. 62:191-219; May, 1993, TIBTECH 11(5):155-215. Methods commonly known in the art of recombinant DNA technology which can be used are described in Ausubel et al., (eds), 1993, Current Protocols in Molecular Biology, John Wiley & Sons, NY; and Kriegler, 1990, Gene Transfer and Expression, A Laboratory Manual, Stockton Press, NY.

In a preferred aspect, the Therapeutic comprises a B14R nucleic acid that is part of an expression vector that expresses a B14R protein or fragment or derivative (for example, chimeric protein) thereof in a suitable host. In particular, such a nucleic acid has a promoter operably linked to the B14R coding region, said promoter being inducible or constitutive, homologous or heterologous, and, optionally, tissue-specific. In another particular embodiment, a nucleic acid molecule is used in which the B14R coding sequences and any other desired sequences are flanked by regions that promote homologous recombination at a desired site in the genome, thus providing for intrachromosomal expression of the B14R nucleic acid, as described (Koller and Smithies, 1989, Proc. Natl. Acad. Sci. USA 86:8932-8935; Zijlstra et al., 1989, Nature 342:435-438).

Delivery of the nucleic acid into a patient may be either direct, in which case the patient is directly exposed to the nucleic acid or nucleic acid-carrying vector, or indirect, in which case, cells are first transformed with the nucleic acid in vitro and then transplanted into the patient. These two approaches are known, respectively as in vivo and ex vivo gene therapy.

Numerous techniques are known in the art for the introduction of foreign genes into cells (see e.g. Loeffler and Behr, 1993, Meth. Enzymol. 217:599-618; Cohen et al., 1993, Meth. Enzymol. 217:618-644; Cline, 1985, Pharmac. Ther. 29:69-92).

In an embodiment in which recombinant cells are used in gene therapy, a B14R nucleic acid is introduced into the cells such that they are expressible by the cell or their progeny, and the recombinant cells are then administered in vivo for therapeutic effect. In a specific embodiment, stem or progenitor cells are used. Any stem and/or progenitor cells which can be isolated and maintained in vitro can be potentially used, such as hematopoietic stem cells (HSC), stem cells of epithelial tissues such as the skin and the lining of the gut, embryonic heart muscle cells, liver stem cells (PCT Publication WO 94/08598, dated Apr. 28, 1994), neural stem cells (Stemple and Anderson, 1992, Cell 71:973-985), or epithelial stem cells (ESCs) (Rheinwald, 1980, Meth. Cell Bio. 21A:229; Pittelkow and Scott, 1986, Mayo Clinic Proc. 61:771).

### Proteins, Derivatives and Fragments

The B14 protein and nucleic acid, and derivatives and fragments thereof can be produced by any method known in the art.

For recombinant expression of proteins, and derivatives and fragments, the nucleic acid containing all or a portion of the nucleotide sequence encoding the protein can be inserted into an appropriate expression vector, i.e., a vector that contains the necessary elements for the transcription and translation of the inserted protein coding sequence. In a preferred embodiment, the regulatory elements (e.g., promoter) are heterologous (i.e., not the native gene promoter). Promoters which may be used include the SV40 early promoter (Bernoist and Chambon, 1981, Nature 290:304-310), and the promoter contained in the 3′ long terminal repeat of Rous sarcoma virus (Yamamoto et al., 1980, Cell 22:787-797), among others.

A variety of host-vector systems may be utilized to express the protein coding sequence. These include mammalian cell systems infected with virus (e.g., VACV, adenovirus, etc.); insect cell systems infected with virus (e.g., baculovirus); microorganisms such as yeast containing yeast vectors; or bacteria transformed with bacteriophage, DNA, plasmid DNA, or cosmid DNA.

Once a B14, or derivative or fragment thereof, has been expressed by recombinant DNA technology, it may be isolated and purified by standard methods including chromatography (e.g., ion exchange, affinity, and sizing column chromatography), centrifugation, differential solubility, or by any other standard technique for the purification of proteins. A B14 protein may also be purified by any standard purification method from natural sources.

Alternatively, a B14 protein, analogue fragment or derivative can be synthesized by standard chemical methods known in the art (e.g., see Hunkapiller et al., 1984, Nature 310:105-111).

The Therapeutics of the invention also include derivatives and fragments related to B14. In particular embodiments, the derivative or fragment is functionally active, i.e., capable of exhibiting one or more functional activities associated with a full-length, wild-type B14 protein, e.g., able to inhibit cell NF-κB activation in in vitro and/or in vivo assays. Derivatives or analogs of B14 can be tested for the desired activity by procedures known in the art.

In specific embodiments of the invention, the Therapeutic is a B14 protein that has been chemically linked to a specific chemical moiety. For example, the protein may be phosphorylated, lipidated or glycosylated. Such modifications may be carried out to improve stability, ease of production, shelf life or in vivo half life.

B14 derivatives may be made by altering the B14 sequence by substitutions, additions, or deletions that provide for functionally equivalent molecules. Due to the degeneracy of nucleotide coding sequences, other DNA sequences that encode substantially the same amino acid sequence as a B14R gene may be used in the practice of the present invention. These include nucleotide sequences comprising all or portions of B14R gene that are altered by the substitution of different codons that encode a functionally equivalent amino acid residue within the sequence, thus producing a silent change. Likewise, the B14 derivatives of the invention include those containing, as a primary amino acid sequence, all or part of the amino acid sequence of a B14 protein including altered sequences in which functionally equivalent amino acid residues are substituted for residues within the sequence resulting in a silent change. For example, one or more amino acid residues within the sequence can be substituted by another amino acid of a similar polarity which acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, trytophan and methionine. The polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid.

In a specific embodiment of the invention, proteins consisting of or comprising a fragment of B14 protein consisting of at least 10 (continuous) amino acids of the B14 protein are provided. In other embodiments, the fragment consists of at least 20 or 50 amino acids of the B14/B15 protein. In specific embodiments, such fragments are not larger than 35, 100 or 200 amino acids. Derivatives or fragments of B14R include but are not limited to those molecules comprising regions that are substantially homologous to B14R or fragments thereof (e.g., in various embodiments, at least 60% or 70% or 80% or 90% or 95% identity over an amino acid sequence of identical size with no insertions or deletions considered, or when compared to an aligned sequence in which the alignment is done by a computer homology program known in the art, e.g., the Blast program) or whose encoding nucleic acid is capable of hybridizing to the inverse complement (the inverse complement of a nucleic acid strand has the complementary sequence running in reverse orientation to the strand so that the inverse complement would hybridize without mismatches to the nucleic acid strand; thus, for example, where the coding strand is hybridisable to a nucleic acid with no mismatches between the coding strand and the hybridisable strand, then the inverse complement of the hybridisable strand is identical to the coding strand) of a coding B14R, under high stringency, moderately stringency, or low stringency conditions.

The B14R derivatives and fragments of the invention can be produced by various methods known in the art. The manipulations that result in their production can occur at the gene or protein level. For example, the cloned B14R gene sequence can be modified by any of numerous strategies known in the art (Sambrook et al., 1990, Molecular Cloning, A Laboratory Manual, 2d, ed., Cold Spring Harbor Laboratory, Cold Spring Harbor, N.Y.).

Additionally, the B14 encoding nucleic acid sequence can be mutated in vitro or in vivo to create and/or destroy translation, initiation, and/or termination sequences or to create variations in coding regions and/or form new restriction endonuclease sites or destroy pre-existing ones, to facilitate further in vitro modification. Any technique for mutagenesis known in the art can be used, including chemical mutagenesis, in vitro site-directed mutagenesis (Hutchinson, C., et al., 1978, J. Biol. Chem. 253:6551), use of TAB® linkers (Pharmacia), etc.

Manipulations of the B14 sequence may also be made at the protein level. Included within the scope of the invention are B14 protein fragments or other derivatives that are differentially modified during or after translation, e.g., by glycosylation, acetylation, phosphorylation, etc. Any of numerous chemical modifications may be carried out by known techniques, including specific chemical cleavage by cyanogens bromide, trypsin, oxidation, reduction; etc.

In addition, analogs and fragments of B14 can be chemically synthesized. For example, a peptide corresponding to a portion of a B14 protein that comprises the desired domain, or that mediates the desired activity in vitro, can be synthesized by use of a peptide synthesizer. Furthermore, if desired, non-classical amino acids or chemical amino acid analogues can be introduced as a substitution or addition into the B14 sequence.

In a specific embodiment, the B14 derivative is a chimeric, or fusion, protein comprising a B14 protein or fragment thereof (preferably consisting of at least a domain or motif of the B14 protein, or at least 15, preferably 20, amino acids of the B14 protein) joined at into amino- or carboxyl-terminus via a peptide bond to an amino acid sequence of a different protein. In one embodiment, such a chimeric protein is produced by recombinant expression of a nucleic acid encoding the protein (comprising a B14 coding sequence joined in-frame to a coding sequence for a different protein). Such a chimeric product can be made by ligating the appropriate nucleic acid sequences encoding the desired amino acid sequences to each other by methods known in the art, in the proper coding frame, and expressing the chimeric product by methods commonly known in the art. Alternatively, such a chimeric product may be made by protein synthetic techniques, e.g., by use of a peptide synthesizer. Chimeric genes comprising portions of B14R fused to any heterologous protein-encoding sequences may be constructed. A specific embodiment relates to a chimeric protein comprising a fragment of B14 of at least six amino acids.

### Pharmaceutical Compositions

The invention relates also to pharmaceutical preparations that contain an active ingredient a nucleic acid molecule, fragment or derivative, or a polypeptide molecule, fragment or derivative, or a pharmaceutically acceptable salt thereof, and to processes for their manufacture. These pharmaceutical preparations are for enteral, such as oral and rectal, or topical or parenteral administration to warm-blooded animals, the preparations containing the pharmacological active ingredient on its own or together with a pharmaceutically acceptable carrier. The pharmaceutical preparations contain, for example, from approximately 10% to approximately 80%, preferable from approximately 20% to approximately 60% active ingredient. Pharmaceutical preparations for enteral and parenteral administrations are, for example, those in unit dose forms, such as dragées, tablets, capsules or suppositories, and also ampoules. They are manufactured in a manner known per se, for example by means of conventional mixing, granulating, confectioning, dissolving or lyophilising processes. For example, pharmaceutical preparations for oral use can be obtained by combining the active ingredient with one or more solid carriers, optionally granulating a resulting mixture, and, if desired, processing the mixture or granules, if appropriate with the addition of suitable excipients, to form tablets or dragée cores.

Suitable carriers are especially fillers, such as sugars, for example lactose, saccarose, mannitol or sorbitol, cellulose preparations and/or calcium phosphates, for example tricalcium phosphate or calcium hydrogen phosphate, also binders, such as starch pastes using, for example, corn, wheat, rice or potato starch, gelatine, tragacanth, methylcellulose an/or polyvinylpyrrolidone, and/or, if desired, disintegrators, such as the above-mentioned starches, also carboxymethyl starch, cross-linked polyvinylpyrrolidone, agar, alginic acid or a salt thereof, such as sodium alginate. Additional excipients are especially flow agents, flow conditioners and lubricants, for example silicic acid, talc, stearic acid or salts thereof, such as magnesium or calcium stearate, and/or polyethylene glycol. Dragée cores are provided with suitable, optionally enteric, coatings, there being used inter alia concentrated sugar solutions which may contain gum arabic, talc, polyvinylpyrrolidone, polyethylene glycol and/or titanium dioxide, or coating solutions in suitable organic solvents or solvent mixtures, or, for the production of enteric coatings, solutions of suitable cellulose preparations, such as acetylcellulose phthalate or hydroxypropylmethylcellulose phthalate. Colourings or pigments may be added to the tablets or dragée coatings, for example for identification purposes or to indicate different doses or active ingredient.

Orally administrable pharmaceutical preparations also include dry-filled capsules consisting of gelatine, and also soft, sealed capsules consisting of gelatine and a plasticizer, such as glycerol or sorbitol. The dry-filled capsules may contain the active ingredient in the form of granules, for example in admixture with fillers, such as lactose, binders such as starches, and/or glidants, such as talc or magnesium stearate, and optionally stabilisers. In soft capsules, the active ingredient is preferably dissolved or suspended in suitable liquid excipients, such as fatty oils, paraffin oil or liquid polyethylene glycols, to which stabilisers may also be added.

Suitable rectally administrable pharmaceutical preparations are, for example, suppositories that consist of a combination of the active ingredient and a suppository base. Suitable suppository bases are, for example, natural or synthetic triglycerides, paraffin hydrocarbons, polyethylene glycols or higher alkanols. It is also possible to use gelatin rectal capsules that contain a combination of the active ingredient and a base material. Suitable base materials are, for example, liquid triglycerides, polyethylene glycols or paraffin hydrocarbons.

Pharmaceutical preparations suitable for topical application are especially creams, ointments, foams, tinctures and solutions that contain from approximately 0.5 to approximately 20% active ingredient.

Creams are oil-in-water emulsions that contain more than 50% water. As oily base there are used especially fatty alcohols, for example lauryl, cetyl, or stearyl alcohol, fatty acids, for example palmitic or steric acid, liquid to solid waxes, for example isopropyl myristate, wool wax or beeswax, and/or hydrocarbons, for example petroleum jelly (petrolatum) or paraffin oil. Suitable emulsifiers are surface-active substances having predominantly hydrophilic properties, such as corresponding non-ionic emulsifiers, for example fatty acid esters of polyalcohols or ethylene oxide adducts thereof, such as polyglycerol fatty acid esters of polyoxyethylene sorbitan fatty acid esters (Tweens), and also polyoxyethylene fatty alcohol ethers or fatty acid esters, or corresponding ionic emulsifiers, such as alkali metal salts of fatty alcohol sulfates, for example sodium lauryl sulphate, sodium cetyl sulphate or sodium stearyl sulphate, which are usually used in the presence of fatty alcohols, for example cetyl alcohol or stearyl alcohol. Additives to the aqueous phase are, inter alia agents that reduce the drying out of the creams, for example polyalcohols, such as glycerol, sorbitol, propylene glycol and/or polyethylene glycols, and also preservatives, perfumes, etc.

Ointments are water-in-oil emulsions that contain up to 70%, but preferably from approximately 20% to approximately 50%, water or aqueous phases. Suitable as fatty phase are especially hydrocarbons, for example petroleum jelly, paraffin oil and/or hard paraffins, which, in order to improve the water-binding capacity, preferably contain suitable hydroxyl compounds, such as fatty alcohols or esters thereof, for example cetyl alcohol or wool wax alcohols, or wool wax. Emulsifiers are corresponding lipophilic substances, such as sorbitan fatty acid esters (Spans), for example sorbitan oleate and/or sorbitan isostearate. Additives to the aqueous phase are, inter alia humectants, such as polyalcohols, for example glycerol, propylene glycol, sorbitol and/or polyethylene glycol, and also preservatives, perfumes, etc.

Fatty ointments are anhydrous and contain as base especially hydrocarbons, for example paraffin, petroleum jelly and/or liquid paraffins, also natural or partially synthetic fat, for example coconut fatty acid triglyceride, or preferably hardened oils, for example hydrogenated groundnut oil or castor oil, also fatty acid partial esters of glycerol, for example glycerol mono- and di-stearate, and also, for example, the fatty alcohols increasing the water-absorption capacity, emulsifiers and/or additives mentioned in connection with the ointments.

Pastes are creams and ointments having secretion-absorbing powder constituents, such as metal oxides, for example titanium oxide or zinc oxide, also talcum and/or aluminium silicates, the purpose of which is to bind any moisture or secretions present.

Foams are administered from pressurised containers and are liquid oil-in-water emulsions in aerosol form; halogenated hydrocarbons, such as chlorofluoro-lower alkanes, for example dichlorodifluoromethane and dichlorotetrafluoroethanem, are used as propellants. As oil phase there are used, inter alia, hydrocarbons for example paraggin oil, fatty alcohols, for example cetyle alcohol, fatty acid esters, for example isopropyl myristate, and/or other waxes. As emulsifiers there are used, inter alia, mixtures of emulsifiers having predominantly hydrophilic properties, such as polyoxyethylene sorbitan fatty acid esters (Tweens) and emulsifiers having predominantly lipophilic properties, such as sorbitan fatty acid esters (Spans). The customary additives, such as preservatives, etc., are also added.

Tinctures and solutions generally have an aqueous-ethanolic base to which there are added, inter alia, polyalcohols, for example glycerol, glycols and/or polyethylene glycol, as humectants for reducing evaporation, and fat-restoring substances, such as fatty acid esters with low molecular weight polyethylene glycols, that is to say lipophilic substances that are soluble in the aqueous mixture, as a replacement for the fatty substances removed from the skin by the ethanol, and, if necessary, other adjuncts and additives.

The manufacture of the topically administrable pharmaceutical preparations is effected in a manner known per se, for example by dissolving or suspending the active ingredient in the base or, if necessary, in a portion thereof. When the active ingredient is processed in the form of a solution, it is generally dissolved in one of the two phases before emulsification; when the active ingredient is processed in the form of a suspension, it is mixed with a portion of the base after emulsification and then added to the remainder of the formulation.

For parenteral administration there are especially suitable aqueous solutions of an active ingredient in water-soluble form, and also suspensions of the active ingredient, such as corresponding oily injection suspensions, there being used suitable lipophilic solvents or vehicles, such as fatty oils, for example sesame oil, or synthetic fatty acid esters, for example ethyl oleate or triglycerides, or aqueous injection suspensions that contain viscosity-increasing substances, for example sodium carboxymethylcellulose, sorbitol and/or dextran, and, if desired, stabilisers.

The dosage of the active ingredient depends upon various factors, such as the species of the warm-blooded animal, its age and individual condition and the mode of administration. In normal cases, the approximate daily dose for a warm-blooded animal weighting about 75 kg is estimated to be, in the case of oral administration, from approximately 150 mg to approximately 1500 mg, advantageously in several equal partial doses.

### EXAMPLE 1

In this study, the B14 protein was identified in infected cells using specific antiserum and a VACV mutant lacking the B14R gene (vΔB14) and a revertant in which the gene was re-inserted into the deletion mutant (vB14-rev) were constructed. The mutant virus was compared in vitro and in vivo to the wild type (vB14) and revertant (vB14-rev) controls. Data presented show that although B14 is non-essential for virus replication in cell culture, it promotes VACV virulence in a murine intradermal model (Tscharke & Smith, 1999; Tscharke et al., 2002; Reading & Smith, 2003) and influences the infiltration of cells into the infected lesion. As such, it represents another intracellular virulence factor that is also an immunomodulator.

### Methods

### Cell Culture

Rabbit kidney RK₁₃ cells, African green monkey kidney cells BS-C-1 and CV-1 cells were grown in Dulbecco's modified Eagle's medium (DMEM) supplemented with 10% heat-inactivated foetal bovine serum (FBS) (Harlan Sera-Lab), 50 IU/ml penicillin and 50 μg ml⁻¹ streptomycin (Gibco BRL) and 2 mM L-glutamine (Gibco BRL). HeLa cells were purchased from the European Collection of Cell Cultures.

### Construction of Plasmid Vectors

Wild type or modified B14R gene fragments were produced by PCR using VACV WR genomic DNA as template and then were cloned into pSJH7 (Hughes et al., 1991) via EcoRI and XbaI restriction sites. To produce plasmid pAB14, a DNA fragment containing the left and right flanking regions of the B14R gene but lacking the entire B14R ORF was produced by overlapping PCR. The 5′ fragment was generated with oligonucleotides 5′-GGAATTCCTTCGGTTCAACTGGAGATTA-3′ (L-FA), containing an EcoRI restriction site (underlined), and 5′-AAATGTCAAGATGTACAACCTTACCAATTGCAATTGGAA-3′, containing nucleotides from the 3′ fragment (italics) at the 5′ end. The 3′ fragment was generated with oligonucleotides 5′-GTTGTACATCTTGACATTT-3′, complementary sequence to the 5′ fragment and 5′-GCTCTAGAGCATTGCTACCATTATCTATC-3′ (R-FB), containing an XbaI restriction site (underlined). Based on the overlapping region, these two fragments were joined by PCR using the L-FA and R-FB oligonucleotides to become an FA-FB fragment. To generate pB14-rev, a DNA fragment containing the entire B14R gene and flanking regions was generated with oligonucleotides FA and FB. To construct a C-terminally HA-tagged B14 VACV (vB14-HA), two fragments were generated and then joined by overlapping PCR. The first fragment was produced by PCR with oligonucleotides L-FA and 5′-AACATCGTATGGGTACATATTCATACGCCGGAATATGA-3′, containing partial HA peptide DNA sequence (italics). The second fragment was amplified by PCR with oligonucleotides R-FB and 5′-ATGTACCCATACGATGTTCCAGATTACGCTTGATGAGTTGTACATCTTGA-3′, containing the entire HA peptide DNA sequence (italics). These two fragments were joined with the L-FA and R-FB to become pSJH7-B14-HA.

To express the wild type B14 protein and B14 tagged at the C terminus with HA in mammalian cells, DNA fragments were generated by PCR using pSJH7-B14-HA as template. For wild type B14 expression (pCl-B14), a DNA fragment containing the B14R gene was produced by PCR with oligonucleotides 5′-GGAATTCCATGACGGCCAACTTTAGTACC-3′ (L-B14), containing an EcoRI restriction site (underlined) and 5′-GCTCTAGAGCTCATCAATTCATACGCCGGAA-3′, containing an XbaI restriction site. For expression of C-terminally-HA tagged B14 (pCl-B14-HA), a DNA fragment encoding the HA peptide fused to the 3′ end of B14R gene was generated by PCR with oligonucleotides L-B14 and 5′-GCTCTAGAGCTCATCAAGCGTAATCTGGAAC, containing XbaI restriction site (underlined) and nucleotides for the HA peptide (italics). These two fragments were cloned into the pCl vector (Promega) by EcoRI and XbaI restriction sites to become pCl-B14-HA. The fidelity of the PCR-derived regions from all plasmids was verified by DNA sequencing.

### Construction of Recombinant Viruses

A VACV deletion mutant lacking the B14R gene coding sequences (vΔB14), a revertant virus with these sequences re-introduced (vB14-rev) and a virus containing a B14R gene encoding a C-terminal HA-tag (vB14-HA) were constructed. The modified and wild type B14R gene were inserted into plasmid pSJH7 (Hughes et al., 1991), containing the E. coli guanine xanthine phosphoribosyltransferase gene as a selectable marker (Boyle & Coupar, 1988). The plasmids were then transfected into either VACV WR-infected CV-1 cells (to make vΔB14) or vΔB14-infected cells (to make vB14-rev and vB14-HA) and recombinant viruses were isolated by transient dominant selection (Falkner & Moss, 1990).

### Immunoblotting

Transfected or infected cells were lysed and prepared for immunoblotting as described previously (Parkinson & Smith, 1994).

### Immunofluorescence

Cells used for fluorescent and confocal microscopy were grown on sterilised glass coverslips (borosilicate glass, BDH) in 6-well plates. The cells were washed three times in ice-cold PBS, fixed in 4% PFA in PBS for 60 min at RT and incubated with 0.1% Triton-X100 (Sigma) for 10 min at RT. The samples were blocked with 10% FBS in PBS for 60 min at RT (or 4° C. overnight), incubated with primary Ab at RT for 1 h and washed three times in PBS. Samples were then incubated with FITC- or TRITC-conjugated secondary Ab at RT for 60 min, washed three times with PBS and subsequently mounted in Mowiol-4′,6-diamidino-2-phenylindole medium. Samples were examined with a Zeiss 512 laser scanning confocal microscope and images were reconstructed and processed using Confocal Assistant and Adobe Photoshop software.

### Virus Growth Curves

Monolayers of BS-C-1 or CV-1 cells were infected with either 10 or 0.01 p.f.u. per cell for measurement of one-step or multi-step growth kinetics, respectively. The culture supernatant was removed at the indicated times and centrifuged at 800 g at 4° C. for 10 min to collect detached cells. Attached cells were scraped into DMEM/2•5% FBS, added to the cells collected from the supernatant, frozen and thawed three times and sonicated to obtain the cell-associated virus. Infectious virions were determined by plaque assay on duplicate BS-C-1 cell monolayers.

### Virulence Assays

For murine intranasal models, female BALB/c mice (5 to 6 weeks old) were anesthetized and infected intranasally with the indicated viral dosage in 20 μl of PBS (Williamson et al., 1990). For the murine intradermal model, female C57BL/6 mice (6-8 weeks old) were anesthetized and injected intradermally in the left ear pinnae with either 10⁴ p.f.u./10 μl of PBS per ear as described previously (Tscharke & Smith, 1999).

### Analysis of Cell Populations in Infected Ears by Flow Cytometry

At the indicated times, intradermally infected mice were sacrificed and ears were removed. Cells that had infiltrated into the infected lesion were collected and analysed as described previously (Reading & Smith, 2003; Jacobs et al., 2006). Briefly, cells in the intradermal compartment migrated into RPMI 1640 medium (Gibco) containing 50 IU ml⁻¹ of penicillin and streptomycin (Gibco), 10% FBS and 2-5 mM HEPES, pH 7•4 in a plastic plate. Adherent and non-adherent cells were pooled, washed once with RPMI/10% FBS, once with Tris/NH₄Cl buffer (0•14 M NH₄Cl in 17 mM Tris, pH 7•2) and twice with FC buffer (0•1% BSA, 0.1% NaN₃ in PBS). The dermal cells were identified by staining of surface markers: CD3 (PE anti-CD3, Caltag Laboratories) on T lymphocytes, Ly6-G (PE anti-Ly6-G, Caltag Laboratories) on neutrophils and F4/80 (PE-anti-F4/80, Serotec) on macrophages.

### Statistical Analysis

Student's t-test (two tailed, unpaired) was used to examine the significance of raw data.

### Results

### Computational Analysis of the B14 Protein

The VACV WR B14R gene (accession number JQ1808) was predicted to encode a 17.3 kDa protein (Howard et al., 1991; Smith et al., 1991) without a transmembrane domain or a secretory signal peptide (see www.poxvirus.org). Computational comparisons detected no orthologues outside poxviruses but found very similar proteins predicted to be encoded by many orthopoxviruses including other VACV strains, Variola virus, Camelpox virus, Monkeypox virus, Ectromelia virus and Cowpox virus with 94-98% amino acid identity. The phylogenetic relationships of these proteins are shown in an rooted tree (FIG. 1, group 1) produced from the aligned amino acid sequences. Another group of more distantly related orthopoxvirus proteins, typified by protein B22 from VACV Copenhagen, was identified and had between 41-42% aa identity and 57-61% aa similarity (FIG. 1, group 11). In some other chordopox virus genera, e.q. Yatapoxvirus, Capripoxvirus, Leporipoxvirus and Suipoxvirus, there are proteins related to B14 that have between 30-41 aa % identity and 49-63 aa % similarity (FIG. 1 group III). However, these proteins clustered more closely with group II proteins from orthopoxviruses than with B14 and the group I proteins. For additional analysis of this family of protein see www.poxvirus.org. The B14 family is part of Poxvirus A4/B15 family (PF06225), and interestingly, proteins related to B14 are not found in the Avipoxvirus and Molluscipoxvirus genera (see www.sanger.ac.uk/Software/Pfam).

### Generation of a Mutant VACV Strain WR Lacking Gene B14R

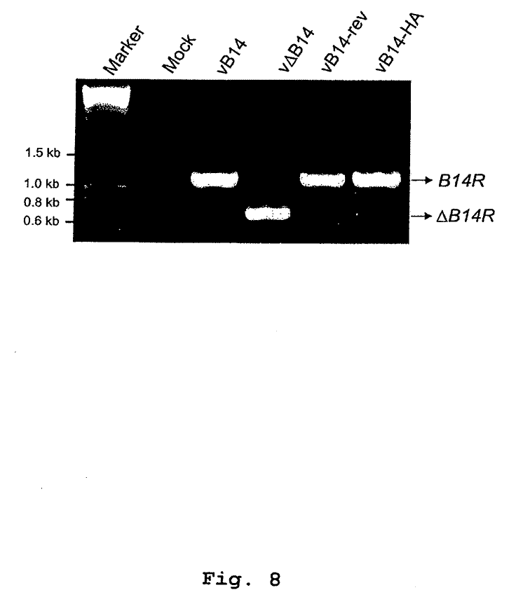

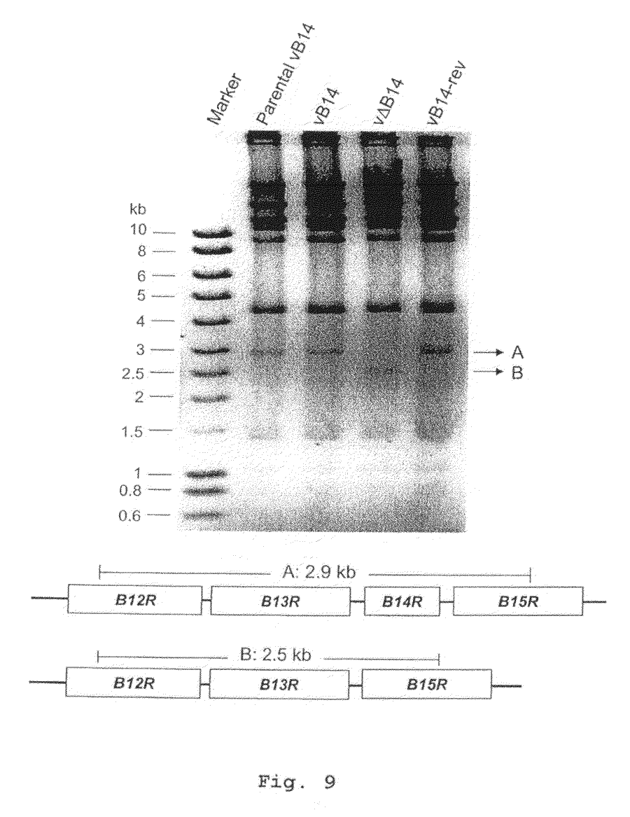

To study the function of B14 protein in virus-infected cells several recombinant viruses were constructed based on VACV strain WR (Methods). These included a plaque purified wild type (vB14), deletion mutant (vΔB14), revertant (vB14-rev) and HA-tagged B14 (vB14-HA). The genomes of these viruses were analysed by PCR and restriction enzyme digestion using DNA extracted from purified intracellular mature virus (IMV). PCR using primers for the B14R gene locus confirmed the presence of the B14R gene in vB14, vB14-rev and vB14-HA and its absence in vΔB14 (FIG. 8). Digestion of genomic DNA with SphI showed similar fragments for all viruses, except that a fragment of approximately 3 kb was reduced in size by 500 bp in vΔB14 compared to controls (FIG. 9), consistent with the loss of the B14R coding sequences. Similar analysis with enzymes HindIII and SalI found no differences outside the B14R locus (data not shown).

### Identification of B14 Expression Using Polyclonal Antiserum

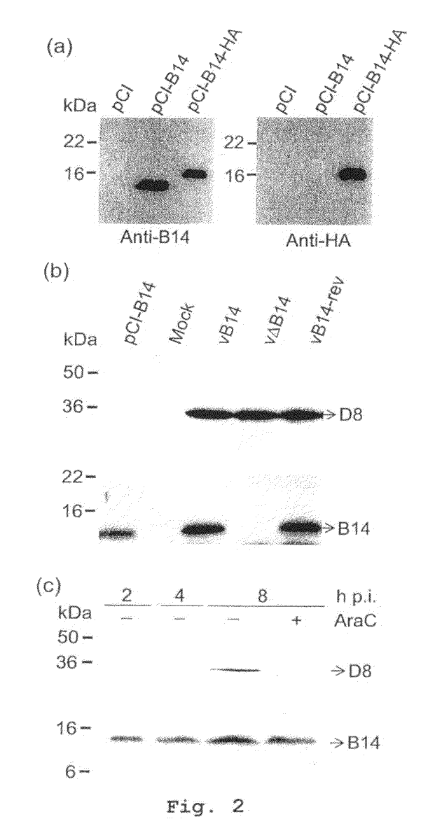

To identify the B14 protein in mammalian cells, the B14R gene was expressed in E. coli with an N-terminal His-tag and the recombinant protein was purified by Ni²⁺ affinity column (data not shown) and used to raise a rabbit anti-B14 polyclonal antibody (Ab). Immunoblot analysis using this Ab identified a 15 kDa protein in extracts of cells transfected with a plasmid containing the B14R ORF driven by the human cytomegalovirus immediate early promoter (FIG. 2a) and in cells infected by VACV WR (FIG. 2b). The specificity of the Ab was confirmed by its recognition of the 15-kDa protein i) only in cells transfected with the B14R ORF but not the empty vector, and ii) in cells infected with wild type and revertant viruses but not vΔB14 (FIG. 2b). The B14 protein was also expressed in mammalian cells with an HA-tag fused to the C terminus. This protein was recognised by an anti-HA mAb and by the anti-B14 Ab, whereas the wild type protein was recognised only by the latter (FIG. 2a). To determine the time of expression of B14 protein during VACV infection, cells were infected in the presence or absence of AraC, an inhibitor of viral DNA replication and late protein expression, and extracts of cells were prepared at different times p.i. and were analyzed by immunoblotting (FIG. 2c). B14 was detected from 2 h p.i., and at all times examined, and was present at higher levels at 8 h p.i. It was still detected in cells in the presence of AraC indicating the protein was made during the early phase of infection. In contrast, AraC blocked the expression of D8, an intracellular mature virion protein known to be expressed late in the VACV life cycle (Niles & Seto, 1988).

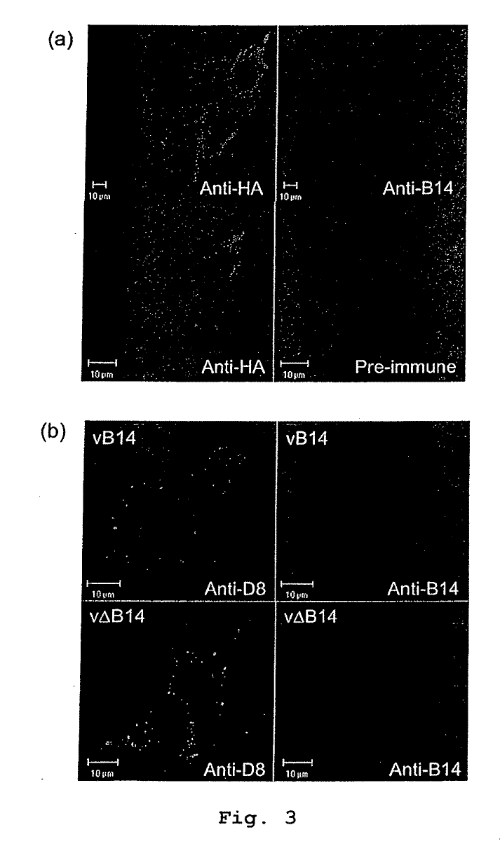

The location of the B14 protein was examined in HeLa cells transfected with the plasmid encoding the HA-tagged B14 protein. Immunofluorescent analysis with the anti-HA mAb and anti-B14 Ab detected a diffuse cytoplasmic staining with no obvious co-localisation to specific organelles and with relatively little staining in the nucleus (FIG. 3a upper panels). Only a weak background signal was detected by pre-immune rabbit serum in the HA-tag positive transfected-cells (FIG. 3a lower right panel). This indicated the specificity of anti-B14 Ab. To compare the location of B14 in transfected cells with those infected by VACV, 8 h p.i. vB14-infected cells were stained with the rabbit Ab and this detected a similar intracellular distribution (FIG. 3b upper right panel). In vΔB14-infected cells, the B14 Ab showed very little staining (FIG. 3b lower right panel). The infection with both viruses was confirmed using a mAb against the VACV D8 protein (FIG. 3b left panels).

### B14 is Non-Essential in Cell Culture

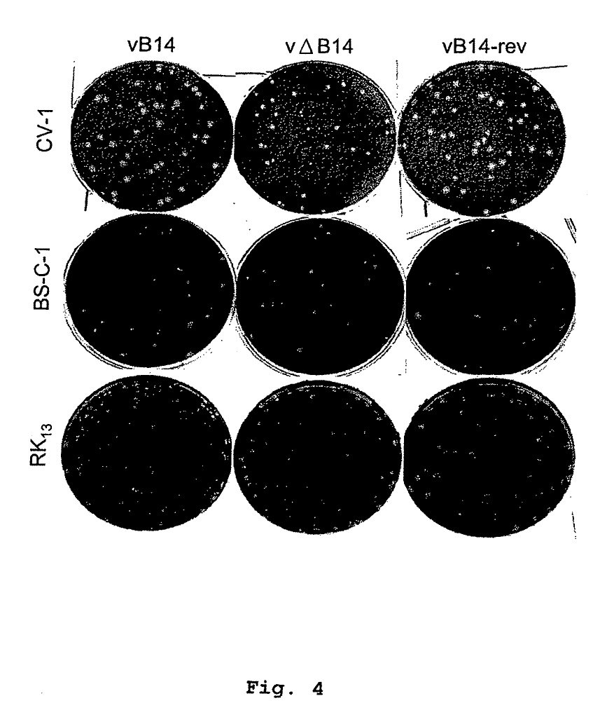

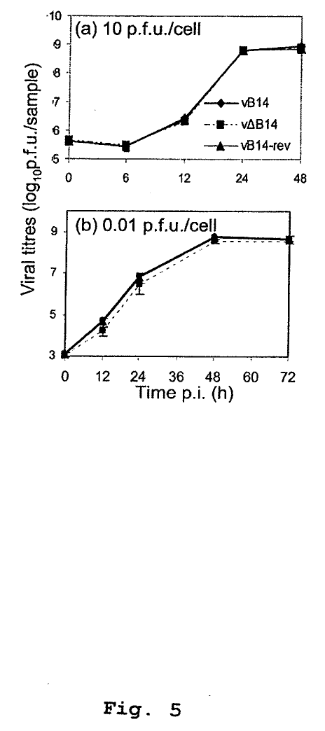

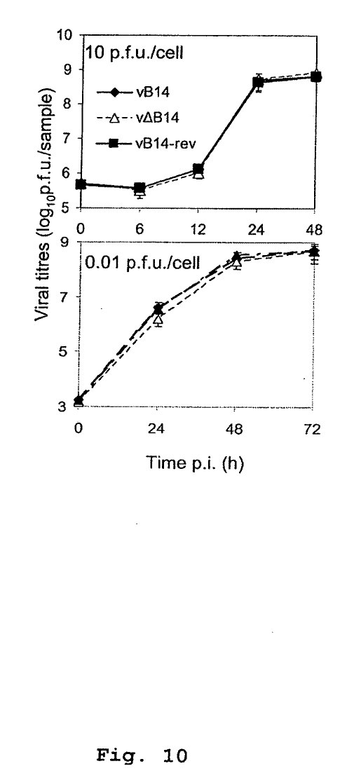

The role of B14 protein in the life cycle of VACV-WR was examined by characterising the phenotype of vΔB14 in cell culture. The plaque size formed by vΔB14 was smaller than that formed by vB14 and vB14-rev in CV-1 cells and slightly smaller in BS-C-1 but not in RK-₁₃ cells (FIG. 4). The reduction in plaque size of CV-1 cells and BS-C-1 was 40% and 20%, respectively (n=20 plaques; P<0.05, Student's t-test). To examine the effect of B14 on the viral life cycle in more detail in vitro, one-step (FIG. 5a) and multi-step (FIG. 5b) growth curves were studied in BS-C-1 and CV-1 cells (FIG. 10). In both cell types vΔB14 showed similar growth kinetics to control viruses after high (10 p.f.u./cell) or low multiplicity (0.01 p.f.u./cell) of infection. Therefore, the basis for the cell type specific plaque size reduction in BS-C-1 and CV-1 cells was not a reduced virus yield.

### The Effect of B14 on Virulence and the Recruitment of Leukocytes

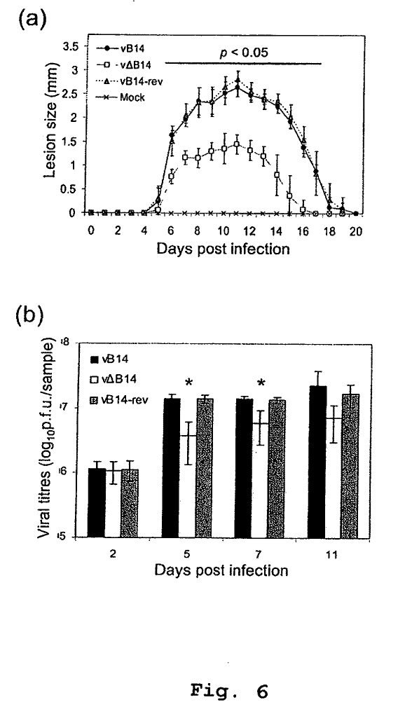

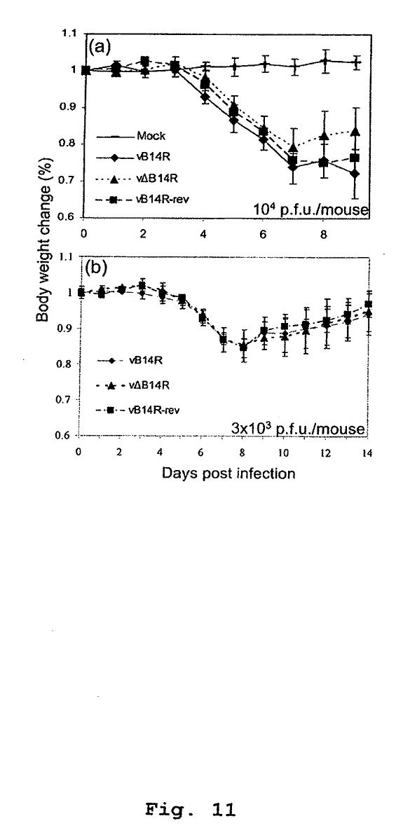

The virulence of vΔB14 was compared to control viruses in 2 murine models. In an intranasal (i.n.) model, no significant difference in weight loss or signs of illness (data not shown) was observed in animals infected with vΔB14 at 10⁴ or 3×10³ p.f.u. compared to control viruses (FIG. 11). However, in an intradermal (i.d.) infection model, vΔB14 induced a significantly (P<0.05) smaller lesion size from day 6 to day 17 p.i. (FIG. 6a). Measurement of infectious virus in infected lesions indicated that although the titres of infectious virus increased equally up to 2 d p.i. thereafter (days 5 and 7) titres were reduced in vΔB14-infected ear lobes compared to controls (FIG. 6b). These observations indicate that B14 enhances virus virulence, at least partly by restricting clearance of virus.

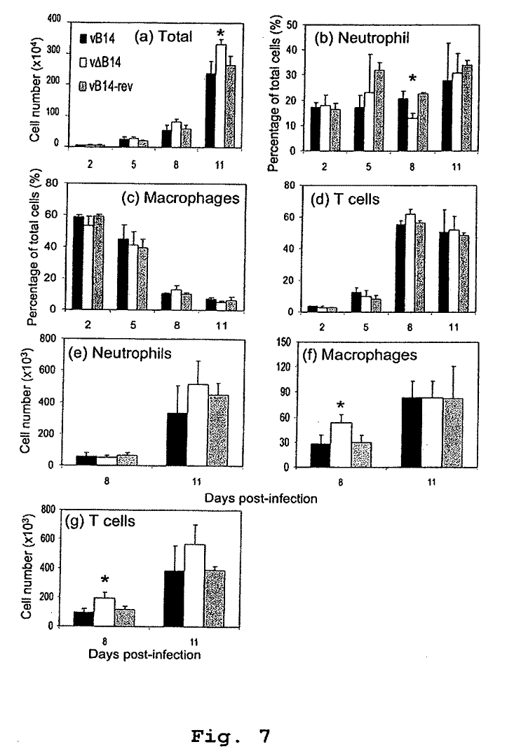

To start to address the mechanism by which B14 affects the outcome of infection in vivo, the cells in the infected ears were extracted, quantified by trypan blue exclusion and identified by flow cytometry (see methods). This revealed a statistically (P<0.05) higher number of cells in vΔB14-infected ears at day 11 p.i. compared to tissue infected by control viruses (FIG. 7a). Interestingly, in vΔB14-infected cells at day 8 p.i., neutrophils represented a reduced percentage of the total cell population compared to controls (FIG. 7b) while the percentage of macrophages (FIG. 7c) and lymphocytes (FIG. 7d) were slightly higher than the controls. However, when the absolute number of each cell type was analysed, rather than the percentage of total cells, the number of macrophages (FIG. 7f) and T cells (FIG. 7g) were significantly higher in ears infected by vΔB14R than in ears infected by controls on day 8 p.i. By this criterion the number of neutrophils was not significantly different between the groups (FIG. 7e). In addition, CD4, CD8, TCRγδ were also examined but showed no significant difference (data not shown). Therefore, the decrease in percentage of neutrophils on day 8 was likely to be attributable to the corresponding increase of macrophages and lymphocytes. A similar result was observed on day 5 p.i. when cells were extracted from ears by digestion with collagenase (Jacobs et al., 2006) (data not shown)

This example provides a characterisation of the B14 protein life cycle. Loss of the B14 protein reduced virus virulence in a murine intradermal but not intranasal model. The attenuation was manifest by a decreased lesion size, reduced virus titres and enhanced recruitment of macrophages and T cells. Collectively, this indicates that B14 is an intracellular immunomodulator.

### EXAMPLE 2

### The Effect of B14 Protein on NF-κB Activity

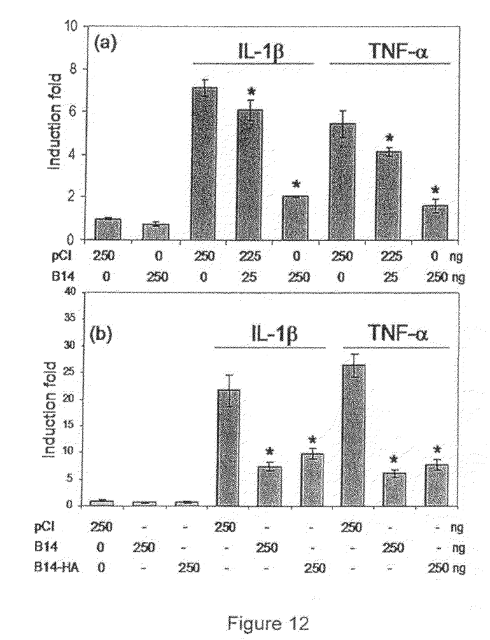

To examine the affect of VACV WR B14 on NF-κB activity, HeLa cells were co-transfected with plasmids i) pCl-B14 (or an empty vector pCl), ii) pNF-κB-Reporter (Sigma) and iii) pSV-B-Gal (Promega) expressing β-galactosidase as a transfection control. At 24 h post-transfection, the cells were stimulated with 100 ng/ml of IL-1β or TNF-α for 6 h (FIG. 4a). Then, extracts were prepared from these cells with Reporter Lysis Buffer (Promega) and the level of cytokine-induced NF-κB gene expression was determined by measuring luciferase activity. In addition, the activity of β-galactosidase from the extracts was measured and then used to normalise the luciferase activity of a corresponding extract. These data were expressed as the mean fold induction relative to the cells expressing empty vector (pCl). Interestingly, these data showed that B14 was able to inhibit the NF-κB activation by either IL-1β or TNF-α in a dose-dependent manner. In addition, a similar inhibition was observed with B14-HA (FIG. 12b). Therefore, the C-terminal HA tag did not alter the function of B14 in this assay.

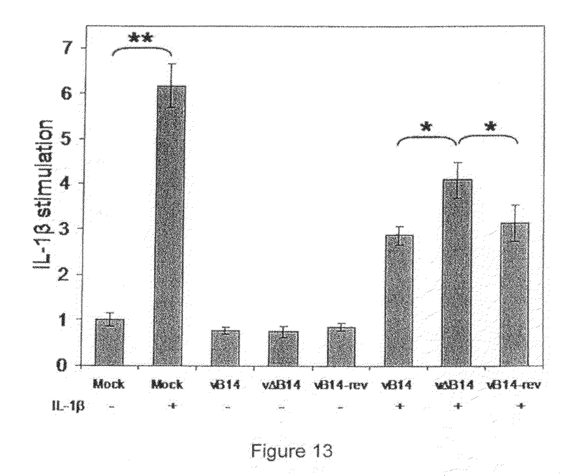

To investigate further the inhibitory effect of B14 on NF-κB-induced gene expression, the pNF-κB-Reporter-transfected cells were infected by the indicated VACVs. At 24 h post-transfection, the HeLa cells were seeded into 24 well plates, incubated overnight and subsequently infected with vB14, vΔB14 or vB14-rev (1 p.f.u/cell). At 2 h p.i., the infected cells were treated with 100 ng/ml of IL-1β for 6 h and then cell extracts were prepared and luciferase activity was measured. In addition, the protein content of the extracts was measured and then used to normalise the luciferase activity of a corresponding extract. Data are expressed as the mean fold induction relative to the mock infection. Compared to mock-infected cells, infection by any of the viruses caused inhibition of NF-κB activity in response to IL-1 stimulation, but notably this inhibition was significantly reduced in vΔB14-infected cells compared to infection with the control viruses (FIG. 13). The significant different (n=3; P<0.05, Student's t-test) between vΔB14 and the controls indicates that B14 also inhibits cytokine-induced NF-κB activation during VACV infection. Also, the fact that vΔB14 infection still caused some inhibition of IL-1-induced NF-κB activation suggests that VACV encodes at least one other inhibitor of NF-κB-induced gene expression.

In summary, B14 inhibited both IL-1β and TNF-α-induced NF-κB activation. These two signalling pathways converge at the IKK complex that phosphorylates IκBα. Consequently, phosphorylated IκBα becomes ubiquitinated and then degraded via the proteosome system. Interestingly, after 4 h p.i., VACV strain COP infection can prevent TNF-α-induced degradation of phosphorylated IκBα and NF-κB activation (Oie & Pickup, 2001). Furthermore, in the same report, CPXV infection was shown to not block completely the phosphorylation of IκBα induced by either TNF or okadaic acid. An interpretation of these data is that B14 interferes with the degradation of phosphorylated IκBα and consequently inhibits NF-κB activation.

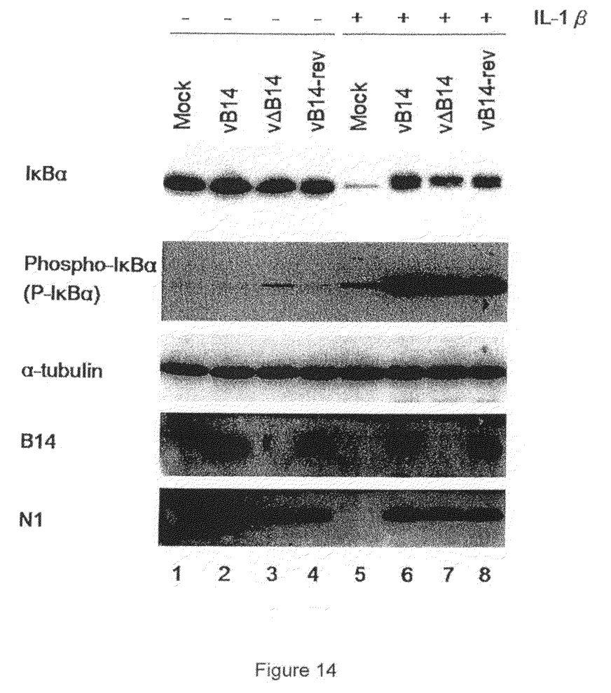

To examine the hypothesis that B14 prevents degradation of phosphorylated IκBα (P-IκBα) and thereby inhibit NF-κB activation, HeLa cells were infected with vB14, vΔB14 or vB14-rev (2 p.f.u./cell) and at 2 h p.i., the infected cells were stimulated with 20 ng/ml of IL-1β (IL-1) or TNF-α for 20 min. Cytoplasmic factions were prepared, resolved by SDS-PAGE (15% gel) and analysed by immunoblotting with anti-IκBα (Santa-Cruz), anti-P-IκBα (Cell Signaling), anti-α-tubulin (Upstate), Abs, anti-serum to B14 or VACV protein N1 (Bartlett et al., 2002) (FIG. 14).

Immunoblotting showed that the level of α-tubulin was indistinguishable in all lanes, suggesting an equal amount of total protein was analysed. In addition, intracellular early VACV protein N1 (Bartlett et al., 2002) was present in the extracts of the cells infected by each virus (bottom panel lane 2-4 & 6-8), whereas B14 was present in vB14 and v-B14-rev infected cells only but not those infected by vΔB14 (FIG. 14). In addition, these two VACV proteins were absent in mock-infected cells. In addition, the expression of B14 and N1 were not affected by stimulation with IL-1β.

In non-stimulated cells there was no difference in the level of IκBα after infection with any virus, nor between infected and mock-infected cells (top panel lanes 1-4). However the level of P-IκBα was increased in cells infected with vΔB14, compared to mock-infected cells or cells infected with vB14 and vB14-rev. This suggests that B14 may inhibit IKK kinase activity partially under these conditions.

After IL-1-stimulation there was a great reduction in IκBα in mock-infected cells (lane 5) compared to unstimulated cells (lane 1). However, the levels of IκBα were increased in infected cells (lanes 6-8) compared to mock-infected cells (lane 5), indicating that VACV infection was preventing degradation of IκBα. Interestingly, the anti-IκBα Ab detected 2 closely migrating bands in the infected cells that had been stimulated with IL-1β (lanes 6-8). The lower band co-migrated with IκBα from unstimulated cells, whereas the upper band showed slightly higher molecular mass, suggesting it to be modified IκBα, for example P-IκBα. Notably, the ratio of the two bands was different in vΔB14-infected cells (lane 7) compared to controls (lane 6 & 8) and there was a significant decrease in the lower band in lane 7. These findings suggested that P-IκBα was present in VACV-infected lysates and that IκBα was degraded more efficiently in the absence of B14.

In addition, the presence of P-IκBα was confirmed with the anti-P-IκBα Ab and showed that VACV infection caused a greater increase in this form compared to mock-infected cells (lane 1). These data indicated that the IL-1-induced degradation of IκBα was highly inhibited by the infection of vB14 and vB14-rev but only partially blocked by the vΔB14 infection. This indicates that B14 affects IκBα degradation and that other VACV protein(s) also contribute to this inhibition of cytokine-induced IκBα degradation.

Although there was a reduced amount of IκBα in the vΔB14-infected cells there was a similar level of P-IκBα after infected with each virus (FIG. 14; Lane 5-8). This indicates that the loss of B14 enhanced the degradation of IκBα but also that infection by vΔB14 may induce higher IKK activity to sustain the same level of P-IκBα. This is supported by higher level of P-IκBα in vΔB14-infected cells compared to controls in the non-stimulated cells. These findings demonstrate that B14 contributes to the inhibition of IL-1-induced IκBα degradation and further implies that B14 impairs IKK activity during virus infection. In addition, the partially inhibited IκBα degradation shows that B14 is one of the viral inhibitors responsible for the inhibition.

In summary, B14 may inhibit NF-κB activity through regulating cytokine-induced IκBα degradation in VACV-infected cells. Furthermore, B14 contributes to regulate IKK activity during VACV infection.

B14 Interacts with the IKK Complex

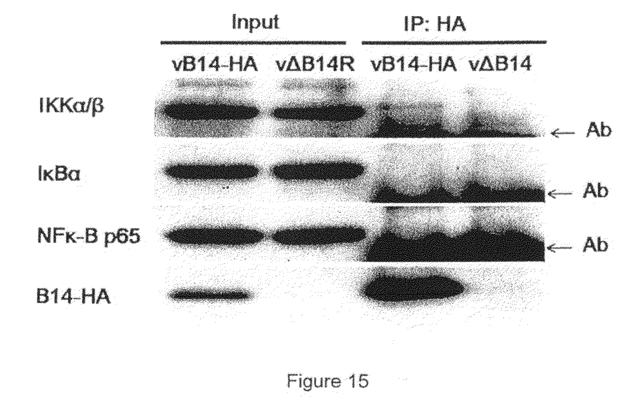

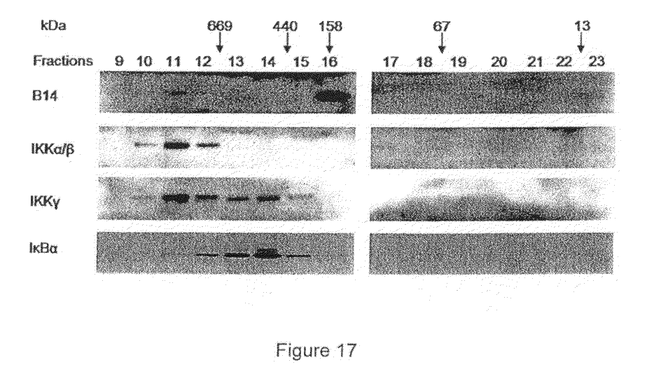

The interaction of B14 with the IKK complex was examined by immunoprecipitation (FIGS. 15 & 16) and gel filtration (FIG. 17).

### Immunoprecipitation

To investigate if B14 might interact with the IKK complex, HeLa cells were infected with vB14-HA or vΔB14 and at 4 h p.i. cytoplasmic extracts were prepared. B14-HA was immunoprecipitated with anti-HA mAb and the immune complex was resolved by SDS-PAGE (12% gel) and then analysed by immunoblotting with Abs against IKKα/β, IκBα, NF-κB (p65) or HA. FIG. 15 shows that anti-HA mAb precipitated B14-HA and IKKα/β from the vB14-HA infected cell lysates but not from the vΔB14-infected lysates. In contrast, IκBα and NF-κB p65 subunit were not found in the immune complex. These findings suggest that B14 interacts specifically with IKKα/β.

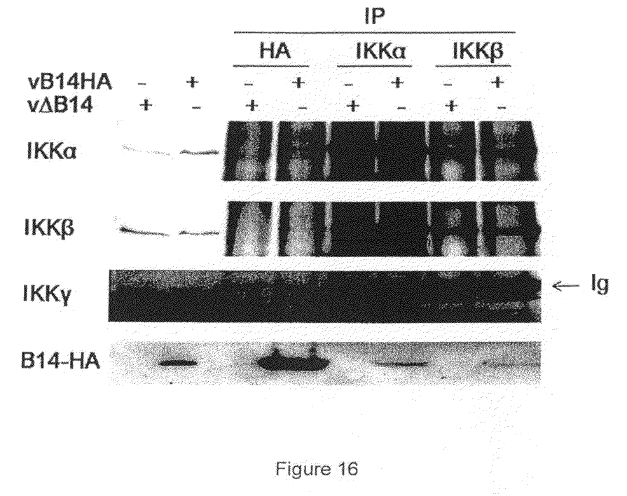

The interaction between B14 and IKKα/β was investigated further by immunoprecipitation with anti-IKKα, anti-IKKβ or anti-HA Abs. IKKα, β and γ were co-immunoprecipitated with B14-HA by anti-HA Ab (FIG. 16). This suggests that B14 is associated with the IKK complex. Similarly, immunoprecipitation with the anti-IKKα/β Abs precipitated B14-HA (FIG. 17). Taken together, these data indicate that B14 interacts with the IKK complex but not with the NF-κB complex.

B14 Co-Purified with the IKK Complex

To examine the interaction between the IKK complex and B14 by an additional method, virus-infected cell lysates were prepared and fractionated according to the size of protein complexes by gel filtration. Fractions from the column were then analysed by immunoblotting (FIG. 17). IKKα, β, and λ were detected mainly in fractions 10-12 and molecular mass markers indicated that these fractions represented a large complex of around 700 kDa, which is the expected size of the IKK complex (DiDonato et al., 1997). Although B14 was identified mainly in fraction 16 (approximately 158 kDa, a small proportion of B14 was also present in fraction 11, the same fraction containing the IKK complex. Notably, IκBα were not detected in the any of the fractions where B14 was present. Taken together, B14 co-purified with the IKK complex but not with the NF-κB complex, and this is consistent with other findings, showing a physical interaction between the IKK complex and B14.

Lastly, it is interesting that the majority of B14 was present as a 158-kDa complex in VACV-infected cells, much larger than the mass of monomeric B14 (17.3 kDa) and the observed migration of denatured B14 on SDS-PAGE (15 kDa). Furthermore, there was no monomeric B14 found in VACV-infected lysates by gel filtration analysis (FIG. 17). Consistent with these findings, it was notable that recombinant B14 protein made in E. coli was also found to be oligomeric. It is possible that the B14 protein is the only protein in the 158-kDa complex alternatively B14 may be complexed with other as yet unidentified cellular or viral proteins.

### Summary

The negative effect of B14 on NF-κB activity was characterised in VACV-infected cells and in transfected cells. In VACV-infected cells, B14 impaired cytokine-induced IκBα degradation but did not bind to IκBα or NF-κB. Instead B14 interacted with the IKK complex and co-purified and co-precipitated with this complex. The interaction with the IKK complex suggests that this is a mechanism by which B14 may modulate IKK activity. Reduced IKK activity would cause reduced P-IκBα and consequently inhibition of NF-κB-induced gene expression.

In both infected and transfected cells, B14 was predominantly cytoplasmic, indicating that other VACV proteins do not influence the location of this protein. Under the conditions examined, there was no apparent co-localisation with specific organelles, although a weak nuclear staining was also apparent. Active nuclear import is dependent on a nuclear localisation signal or carrier protein(s) such as importin that shuttle between the cytoplasm and nucleus. Alternatively, small proteins (less than 2040 kDa) can pass through nuclear pores by passive diffusion (Macara, 2001; Xu & Massague, 2004). Therefore, the presence of some B14 in the nucleus might be via an unknown active transport system or simply by passive diffusion into the nucleus due to the small molecular mass of BI4

B14 is a nonessential protein as shown by the isolation of a viable deletion mutant (vΔB14) lacking this gene and the in vitro characterisation of vΔB14. This is consistent with previous a finding using a VACV without several genes including B14R (Perkus et al., 1991). Although the mutant displayed reduced plaque size compared to wild type and revertant control viruses in BS-C-1 and CV-1 cells, the plaque formed by each virus was undistinguishable in size in RK₁₃ cells. However, vΔB14 failed to show a significant decrease in growth kinetics, even in BS-C-1 and CV-1 cells. The major factor determining plaque size is the ability to form virus-tipped actin tails beneath CEV particles at the cell surface that drive surface virions into surrounding cells, for review see (Smith et al., 2002). However, an analysis of actin tail formation in cells infected by vΔB14 showed no difference from controls (data not shown). Other VACV proteins affect the plaque size or morphology without inhibiting actin tail formation. For instance, a VACV strain lacking kelch-like protein C2 produced plaques with a distinct morphology but the same size (Pires de Miranda et al., 2003) and either the deletion (Parkinson et al., 1995) or over-expression (Sanderson et al., 1996) of the A38 protein reduced plaque size. Therefore, B14 is not required for the viral replication and the basis for the restricted plaque size is not due to a cell-line specific defect in viral growth.

In vivo analysis showed that B14 contributed to virus virulence in a murine i.d. (local infection) but not i.n. (systemic infection) model. Previously, other VACV immunomodulators were shown to exhibit a phenotype in either one model or the other and this may be explained by the different inflammatory responses following infection with VACV via the i.n. and i.d. infections (Tscharke et al, 2002; Reading & Smith, 2003). Analysis of infectious virus titres in infected dermal tissue showed that by 2 d p.i. the virus titres had increased for all viruses to a similar extent; therefore, there was no replicative defect associated with loss of B14 in vivo. However, subsequently (d 5 and 7 p.i.) the titres of virus in ears infected with vΔB14 was reduced compared to controls. This finding suggested that B14 impaired the ability of the host response to control virus growth, and so in its absence virus titres were lower and lesions sizes were smaller.

To examine the effect of B14 in vivo further, cells that were present in the infected lesions were extracted and analysed by staining with specific Ab and FACS analysis. Although there was a trend showing an increased infiltration of cells into the vΔB14-infected ears on day 5 and 8 p.i. this was only significantly different from controls at day 11 p.i. The absolute number of neutrophils present in infected lesions was not altered at any time point, but the number of macrophages and T cells rose significantly at day 8 p.i. in vΔB14-infected lesions compared to controls. This increase in leukocytes indicates an enhanced host inflammatory response in the absence of B14, and this enhanced inflammatory response may contribute to the size restriction and early resolution of the resulting lesion.

The recruitment of leukocytes to the skin is dependent on the expression of chemokines and cytokines (Gillitzer & Goebeler, 2001; Schon et al., 2003). Among these mediators, chemokines CCL2 and CCL3 (formerly monocyte chemoattractant protein-1 and macrophage inflammatory protein-1alpha, respectively) play important roles in recruiting T cells and macrophages (Kunstfeld et al., 1998; von Stebut et al, 2003). Therefore, the production of such chemokines early after infection will affect the cellular infiltrate. Infection by vΔB14 caused a greater infiltration of leukocytes into infected tissues on day 5-11 p.i. compared to infection by control viruses (although this was only significantly different on day 11). Notably these infiltrates contained increased numbers of T cells and macrophages (day 8 p.i.). Taken together, these findings suggest that B14 may modulate the early inflammatory response, possibly by affecting the expression of pro-inflammatory chemokines such as CCL2 and CCL3.

NF-κB has been shown to be an essential modulator of transcription of the following chemokines: CXCL1, -2, -3, -5, -8, -9, -10 and -12, and CCL2, -3, -4, -5, -3 and -17 (Richmond, 2002) and B14 inhibited cytokine-induced NF-κB activation in both uninfected and VACV-infected cells. This suggests that B14 may modulate immune responses by affecting NF-κB-induced gene expression in vivo. This may explain why the profile and amount of infiltrating leukocytes is altered by removing B14 in the intradermal VACV infection model. However, the mechanism by which B14 exerts such immunomodulation remains to be investigated. One hypothesis is that the presence of B14 in infected cells restricts the production of NF-κB-induced cytokines and chemokines, and this leads in turn to a diminished inflammatory response. This may be tested in the future by either ELISAs on homogenates of VACV-infected tissues or microarray analysis of host gene expression in the presence or absence of BI4.

The mechanism by which B14 inhibits NF-κB activation was examined in both infected and uninfected cells. Ectopically expressed B14 inhibited NF-κB gene expression induced by IL-1β or TNF-α, These two signalling pathways converge on the IKK signalling complex leading to activation of NF-κB (Woronicz et al., 1997; Zandi et al., 1997; Sizemore et al., 2002). This implies that B14 inhibits NF-κB activation possibly by targeting to the IKK signalosome and/or downstream events such as IκBα degradation. Other VACV proteins also regulate NF-κB activation. For instance, N1 was reported to inhibit NF-κB activation by targeting the IKK complex (DiPerna et al., 2004). In contrast, VACV-encoded toll-like receptor inhibitors, A46 and A52, affected IL-1- but not TNF-induced signalling pathways upstream of the IKK complex (Bowie et al., 2000; Harte et al., 2003; Stack et al., 2005). These findings indicated that over-expressed N1, A46 and A52 can inhibit cytokine-induced IKK activity in cell culture. However, the cytokine stimulated phosphorylation of IκBα was observed in VACV infected cells in this study. Taken together, N1, A46 and A52 remain to be further characterised in a VACV infection condition.

Two hours after VACV infection, the B14 deletion mutant showed notably higher level of P-IκBα but similar level of IκBα compared to control viruses by immunoblotting. In addition, VACV-expressed B14 was found to be associated with the IKK complex by immunoprecipitation and gel filtration. These findings show that B14 interacts with IKK and suggest that it might modify IKK function, such as downregulating the phosphorylation of IκBα, It was also noted that VACV infection did not change the overall size of IKK complex, suggesting that B14 is unable to interrupt the formation of IKK signalosome or induce its disassembly. Additional experimentation is needed to determine the mechanism by which the interaction of B14 with the IKK complex inhibits activation of NF-κB.

The higher level of P-IκBα in cells infected by vΔB14 may have relevance to the formation of smaller plaques by this virus on some cell types. The higher level of P-IκBα suggests that the activity of NF-κB might be higher in the vΔB14-infected cells early during the infection. If so this might affect plaque formation formed by vΔB14.

In VACV-infected cells it was shown that the cytokine-induced P-IκBα was stabilised by 2 h p.i. and, interestingly, that the loss of B14 partially restored the degradation of IκBα after IL-1β or TNF-α stimulation (data not shown). This suggests that B14 is one of the viral inhibitors responsible for inhibiting the degradation of IκBα but there must be others. Similarly, CPXV also inhibits the cytokine NF-κB activation by interfering with the degradation of IκBα, even after it has been phosphorylated at 4 h p.i. (Oie & Pickup, 2001). In previous reports, a K1 deletion mutant induced the degradation of IκBα and consequently the activation of IκBα in RK13 cells (Shisler & Jin, 2004).

The degradation of IκBα is mediated by a ubiquitin-proteasome pathway (Chen, 2005). After the IKK complex phosphorylates IκBα, an IκBα-ubiquitin ligase complex recognises the P-IκBα via the β-TrCP protein and ubiquitinates P-IκBα (Kroll et al., 1999; Spencer et al., 1999). In the literature, the human immunodeficiency virus type 1 Vpu protein was shown to inhibit NF-κB activation by targeting β-TRCP degradation of IκBα (Akari et al., 2001; Bour et al., 2001), In addition, a Yersinia pestis virulence factor YopJ inhibits NF-κB activation by removing ubquitin molecules attached to a lysine 48 residue on IκBα, and consequently blocks the proteolysis of IκBα (Zhou et al., 2005). These findings suggest that the inhibitory effect of B14 on the process of P-IκBα may be caused by interfering with the function of β-TrCP. In addition, perhaps B14 acts as a de-ubiquinase or contributes to de-ubiquitination that affects both IKK activity and IκBα. These possible mechanisms for the function of B14 remain to be investigated.

References cited herein are hereby incorporated by reference in their entirety.

### REFERENCES

Alcami, A. & Smith, G. L. (1992). A soluble receptor for interleukin-1 beta encoded by vaccinia virus: a novel mechanism of virus modulation of the host response to infection. Cell 71, 153-167.
Alcami, A. & Smith, G. L. (1996). A mechanism for the inhibition of fever by a virus. Proc Natl Acad Sci USA 93, 11029-11034.
Bartlett, N., Symons, J. A., Tscharke, D. C. & Smith, G. L. (2002) The vaccinia virus Ni L protein is an intracellular homodimer that promotes virulence, J Gen Virol 83, 1965-1976
Bowie, A., Kiss-Toth, K, Symons, J. A., Smith, G. L., Dower, S. K. & O L. A. (2000). A46R and A52R from vaccinia virus are antagonists of host IL-i and toll-like receptor signaling. Proc Natl Acad Sci USA 97, 10162-10167
Boyle, D. B. & Coupar, B. E. (1988). A dominant selectable marker for the construction of recombinant poxviruses. Gene 65, 123-128.
Chen, Z. J. (2005). Ubiquitin signalling in the NF-kappaB pathway. Nat Cell Biol 7, 758-765
DiDonato, J. A., Hayakawa, M., Rothwarf, D. M., Zandi, E. & Karin, M. (1997). A cytokine-responsive IkappaB kinase that activates the transcription factor NF-kappaB. Nature 388, 548-554
DiPerna, G., Stack, J., Bowie, A. G., Boyd, A., Kotwal, G., Zhang, Z., Arvikar, S., Latz, E., Fitzgerald, K. a. & Marshall, W. L. (2004). Poxvirus protein N1L targets the I-kappaB kinase complex, inhibits signaling to NF-kappaB by the tumor necrosis factor superfamily of receptors, and inhibits NF-kappaB and IRF3 signaling by toll-like receptors. J Biol Chem 279, 36570-36578
Dobbelstein, M. & Shenk, T. (1996). Protection against apoptosis by the vaccinia virus SPI-2 (B13R) gene product. J Virol 70, 6479-6485.
Falkner, F. G. & Moss, B. (1990). Transient dominant selection of recombinant vaccinia viruses. J Virol 64, 3108-3111.
Gillard, S., Spehner, D., Drillien, R. & Kirn, A. (1986). Localisation and sequence of a vaccinia virus gene required for multiplication in human cells. Proc Natl Acad Sci USA 83, 5573-5577.
Gillitzer, R. & Goebeler, M. (2001). Chemokines in cutaneous wound healing. J Leukoc Biol 69, 513-521.
Goebel, S. J., Johnson, G. P., Perkus, M. E., Davis, S. W., Winslow, J. P. & Paoletti, E. (1990). The complete DNA sequence of vaccinia virus. Virology 179, 247-266, 517-263.
Gubser, C. & Smith, G. L. (2002). The sequence of camelpoxvirus shows it is most closely related to variola virus, the cause of smallpox. J Gen Virol 83, 855-872.
Gubser, C., Hue, S., Kellam, P. & Smith, G. L. (2004). Poxvirus genomes: a phylogenetic analysis. J Gen Virol 85, 105-117.
Harte, M. T., Haga, I. R., Maloney, G., Gray, P., Reading, P. C., Bartlett, N. W., Smith, G. L., Bowie, A. & O'Neill, L. A. (2003). The poxvirus protein A52R targets Toll-like receptor signaling complexes to suppress host defense. J Exp Med 197, 343-351.
Howard, S. T., Chan, Y. S. & Smith, G. L. (1991). Vaccinia virus homologues of the Shope fibroma virus inverted terminal repeat proteins and a discontinuous ORF related to the tumor necrosis factor receptor family. Virology 180, 633-647.
Hughes, S. J., Johnston, L. H., de Carlos, A. & Smith, G. L. (1991). Vaccinia virus encodes an active thymidylate kinase that complements a cdc8 mutant of Saccharomyces cerevisiae. J Biol Chem 266, 20103-20109.
Jacobs, N., Chen, R. A.-J., Gubser, C., Najarro, P. & Smith, G. L. (2006). Intradermal immune response after infection with Vaccinia virus. J Gen Virol submitted.
Kettle, S., Alcami, A., Khanna, A., Ehret, R., Jassoy, C. & Smith, G. L. (1997). Vaccinia virus serpin B13R(SPI-2) inhibits interleukin-1beta-converting enzyme and protects virus-infected cells from TNF- and Fas-mediated apoptosis, but does not prevent IL-1 beta-induced fever. J Gen Virol 78, 677-685.
Kroll, M., Margottin, F., Kohl, A., Renard, P., Durand, H., Concordet, J. P., Bachelerie, F., Arenzana-Sisdedos, F. & Benarous, R. (1999). Inducible degradation of IkappaBalpha by the preoeasome requires interaction with the F-box protein h-beta TrCP. J biol Chem 274, 7941-7945.
Kunsffeld, R., Lechleitner, S., Wolff, K. & Petzelbauer, P. (1998). MCP-1 and MIP-1alpha are most efficient in recruiting T cells into the skin in vivo. J Invest Dermatol 111, 1040-1044.
Macara, I. G. (2001). Transport into and out of the nucleus. Microbiol. Mol Biol Rev 65, 570-594, table of contents.
Moss, B. (2001). Poxyiridae: the viruses and their replication. In Virology, 4th edn, pp. 2849-2883. Edited by B. N. Fields, D. M. Knipe, P. M. Howley, R. M. Chanock, J. Melnick, T. P. Monath, B. Roizman & S. E. Straus. Philadelphia: Lippincott-Raven Publishers.
Niles, E. G. & Seto, J. (1988). Vaccinia virus gene D8 encodes a virion transmembrane protein. J Virol 62, 3772-3778.
Parkinson, J. E. & Smith, G. L. (1994). Vaccinia virus gene A36R encodes a M(r) 43-50 K protein on the surface of extracellular enveloped virus. Virology 204, 376-390.
Perkus, M. E., Goebel, S. J., Davis, S. W., Johnson, G. P., Norton, E. K. and Paoletti, E. (1991) Deletion of 55 open reading frames from the termini of vaccinia virus. Virology, 180, 406-410.
Pires de Miranda, M., Reading, P. C., Tscharke, D. C., Murphy, B. J. & Smith, G. L. (2003). The vaccinia virus kelch-like protein C2L affects calcium-independent adhesion to the extracellular matrix and inflammation in a murine intradermal model. J Gen Virol 84, 2459-2471.
Oie, K. K. & Pickup, D. j. (2001). Cowpox virus and other members of the orthopoxvirus genus interfere with the regulation of KF-kappaB activation. Virology 288, 175-187
Pires de Miranda, M., Reading, P. C., Tscharke, D. C., Murphy, B. J. & Smith, G. L. (2003). The vaccinia virus kelch-like protein C2L affects calcium-independent adhesion to the extracellular matrix and inflammation in a murine intradermal model. J Gen Virol 84, 2459-2471
Reading, P. C. & Smith, G. L. (2003). A kinetic analysis of immune mediators in the lungs of mice infected with vaccinia virus and comparison with intradermal infection. J Gen Virol 84, 1973-1983.
Richmond, A. (2002). Nf-kappa B, chemokine gene transcription and tumour growth. Nat Rev immunol 2, 664-674.
Schon, M. P., Zollner, T. M. & Boehncke, W. H. (2003). The molecular basis of lymphocyte recruitment to the skin: clues for pathogenesis and selective therapies of inflammatory disorders. J Invest Dermatol 121, 951-962.
Seet, B. T., Johnston, J. B., Brunetti, C. R., Barrett, J. W., Everett, H., Cameron, C., Sypula, J., Nazarian, S. H., Lucas, A. & McFadden, G. (2003). Poxviruses and immune evasion. Annu Rev Immunol 21, 377-423.
Smith, G. L. & McFadden, G. (2002). Smallpox: anything to declare? Nat Rev Immunol 2, 521-527.
Smith, G. L., Chan, Y. S. & Howard, S. T. (1991). Nucleotide sequence of 42 kbp of vaccinia virus strain WR from near the right inverted terminal repeat. J Gen Virol 72, 1349-1376.
Spriggs, M. K., Hruby, D. E., Maliszewski, C. R., Pickup, D. J., Sims, J. E., Buller, R. M. & VanSlyke, J. (1992). Vaccinia and cowpoxviruses encode a novel secreted interleukin-1-binding protein. Cell 71, 145-152.
Tscharke, D. C. & Smith, G. L. (1999). A model for vaccinia virus pathogenesis and immunity based on intradermal injection of mouse ear pinnae. J Gen Virol 80, 2751-2755.
Tscharke, D. C., Reading, P. C. & Smith, G. L. (2002). Dermal infection with vaccinia virus reveals roles for virus proteins not seen using other inoculation routes. J Gen Virol 83, 1977-1986.
Upton, C., Slack, S., Hunter, A. L., Ehlers, A. & Roper, R. L. (2003). Poxvirus orthologous clusters: toward defining the minimum essential poxvirus genome. J Virol 77, 7590-7600.
von Stebut, E., Metz, M., Milon, G., Knop, J. & Maurer, M. (2003). Early macrophage influx to sites of cutaneous granuloma formation is dependent on MIP-1alpha/beta released from neutrophils recruited by mast cell-derived TNFalpha. Blood 101, 210-215.
Williamson, J. D., Reith, R. W., Jeffrey, L. J., Arrand, J. R. & Mackett, M. (1990). Biological characterization of recombinant vaccinia viruses in mice infected by the respiratory route. J Gen Virol 71, 2761-2767.
Xu, L. & Massague, J. (2004). Nucleocytoplasmic shuttling of signal transducers. Nat Rev Mol Cell Biol 5, 209-219.

1. A recombinant poxvirus, wherein the poxvirus genome does not contain a functional gene corresponding to B14R in the WR strain of VACV, for use as a medicament.

2. A recombinant poxvirus as claimed in claim 1 for use as a vaccine against a disease caused by a poxvirus.

3. A recombinant poxvirus as claimed in claim 1 wherein the recombinant poxvirus is an orthopoxvirus or a derivative thereof.

4. A recombinant poxvirus as claimed in claim 1, in which the poxvirus has no coding sequence corresponding to the sequence of B14R in the WR strain of VACV.

5. A recombinant poxvirus as claimed in claim 1, in which the gene corresponding to B14R in the WR strain of VACV is disrupted, mutated or truncated such that its gene product has reduced activity.

6. A recombinant poxvirus as claimed in claim 1, wherein the poxvirus genome comprises a non-poxvirus gene or a fragment of a non-poxvirus gene which gene or fragment encodes an antigen.

7. A vaccine composition comprising a poxvirus as defined in claim 1 and a pharmaceutically suitable carrier.

8. A vaccine kit comprising a composition as claimed in claim 7.

9. A method of vaccinating a subject comprising administering to the subject an immunogenic agent, wherein the immunogenic agent is a poxvirus as defined in claim 1 or a vaccine composition as claimed in claim 7.

10. (canceled)

11. A recombinant poxvirus having a genome comprising a non-poxvirus gene or a fragment of a non-poxvirus gene which gene or fragment encodes an antigen, wherein the poxvirus genome does not comprise a functional gene corresponding to B14R in the WR strain of VACV.

12. A recombinant poxvirus as claimed in claim 11 wherein the poxvirus is an orthopoxvirus or a derivative thereof.

13. A recombinant poxvirus as claimed in claim 11 in which the non-poxvirus gene or gene fragment that encodes an antigen is a non-poxvirus gene or gene fragment against the gene product of which a protective immune response in a subject is desirable.

14. A recombinant poxvirus as claimed in claim 11, for use as a vaccine for the prophylaxis of an infection caused by a pathogenic agent.

15. A recombinant poxvirus as claimed in claim 14 in which the non-poxvirus gene or gene fragment encodes an immunogenic peptide or polypeptide of an infectious pathogen, for example an influenza virus, malaria, HIV, heptitis C virus, hepatitis B virus, herpes viruses, a parasitic pathogen, for example tuberculosis or Leishmaniasis, a protozoan, for example a protozoan that causes ameobic dysentery.

16. A recombinant poxvirus as claimed in claim 11 for use as a vaccine for the prophylaxis or treatment of a disease associated with aberrant cells.

17. A recombinant poxvirus as claimed in claim 16 in which the non-poxvirus gene encodes an antigenic peptide or polypeptide of aberrant cells, for example cancer cells, the elimination or induced quiescence of which is beneficial.

18. A recombinant poxvirus as claimed in claim 11, in which the poxvirus has no coding sequence corresponding to B14R in the WR strain of VACV.

19. A recombinant poxvirus as claimed claim 11, in which the gene corresponding to B14R in the WR strain of VACV, mutated or truncated such that its gene product has reduced activity.

20. A recombinant vaccine composition comprising a poxvirus as defined in claim 11 and a pharmaceutically suitable carrier.

21. A vaccine kit comprising a composition as claimed in claim 20.

22. A method of vaccinating a subject comprising administering to the subject an immunogenic agent, wherein the immunogenic agent is a poxvirus as defined in claim 11 or a vaccine composition as claimed in claim 20.

23. (canceled)

24. (canceled)

25. An isolated nucleic acid molecule comprising a nucleotide sequence corresponding to a poxvirus gene corresponding to B14R in the WR strain of VACV, or a functional fragment or derivative thereof.

26. An isolated polypeptide molecule comprising an amino acid sequence corresponding to a poxvirus protein corresponding to B14 in the WR strain of VACV, or a functional fragment or derivative thereof.

27. An isolated nucleic acid molecule, or a functional fragment or derivative thereof, as claimed in claim 25 or an isolated polypeptide molecule or a functional fragment or derivative thereof, as claimed in claim 26, for use as a medicament.

28. An isolated nucleic acid molecule, or a functional fragment or derivative thereof, or an isolated polypeptide molecule or a functional fragment or derivative thereof, according to claim 25 or claim 26 for use as a medicament against a disease or disorder characterized by undesirable inflammation.

29. An isolated nucleic acid molecule, or a functional fragment or derivative thereof, or an isolated polypeptide molecule, or a functional fragment or derivative thereof, according to claim 25 or claim 26 for use as a medicament against a disease or disorder characterized by undesirable immune activation.

30. An isolated nucleic acid molecule, or a functional fragment or derivative thereof, or an isolated polypeptide molecule, or a functional fragment or derivative thereof, according to claim 25 or claim 26 for use as a medicament against a disease or disorder characterized by undesirable NF-κB activation.

31. (canceled)

32. A pharmaceutical composition comprising an isolated nucleic acid molecule, or a functional fragment or derivative thereof, or an isolated polypeptide molecule, or a functional fragment or derivative thereof, according to claim 25 or claim 26 and a pharmaceutical carrier.

33. A method of inhibiting NF-κB activation in cells comprising providing cells with an effective amount of an isolated polypeptide molecule as claimed in claim 26.

34. A method of treating a disease or disorder characterized by undesirable NF-κB activation, comprising administering a therapeutically effective amount of an isolated nucleic acid molecule, or a functional fragment or derivative thereof, or an isolated polypeptide molecule, or a functional fragment or derivative thereof, according to claim 25 or claim 26 to a subject in need of said treatment.

35. A method of treating a disease or disorder characterized by undesirable inflammation, comprising administering a therapeutically effective amount of an isolated nucleic acid molecule, or a functional fragment or derivative thereof, or an isolated polypeptide molecule, or a functional fragment or derivative thereof, according to claim 25 or claim 26 to a subject in need of said treatment.

36. A method of treating a disease or disorder characterized by undesirable immune activation, comprising administering a therapeutically effective amount of an isolated nucleic acid molecule, or a functional fragment or derivative thereof, or an isolated polypeptide molecule, or a functional fragment or derivative thereof, according to claim 25 or claim 26 to a subject in need of said treatment.
